(19) United States
(12) Patent Application Publication
(10) Pub. No.: US 2023/0165824 A1
(43) Pub. Date: Jun. 1, 2023

(54) METHODS, SYSTEMS AND COMPOSITIONS FOR INHIBITION OF CELLULAR DYSFUNCTION AND CELL DEATH WITH DEUTERATED PUFAs

(71) Applicant: BioJiva LLC, Los Altos, CA (US)

(72) Inventors: Peter Milner, Los Altos, CA (US); Mikhail Sergeevich Schepinov, Oxford (GB)

(21) Appl. No.: 17/902,684

(22) Filed: Sep. 2, 2022

Related U.S. Application Data
(60) Provisional application No. 63/240,751, filed on Sep. 3, 2021.
(60) Provisional application No. 63/253,061, filed on Oct. 6, 2021.
(60) Provisional application No. 63/253,690, filed on Oct. 8, 2021.
(60) Provisional application No. 63/293,219, filed on Dec. 23, 2021.

(51) Int. Cl. A61K 31/202; A61P 43/00

(52) U.S. Cl. CPC A61K 31/202; A61P 43/00

(57) ABSTRACT

Disclosed are methods for treatment of neurodegenerative diseases or inhibiting the progression of neurodegenerative disease. The methods may comprise inhibiting cellular dysfunctionality and subsequent cell death due to cellular accumulation of oxidized polyunsaturated fatty acids (PUFAs) products wherein said accumulation is mediated, at least in part, by impaired enzymatic process(es) that are responsible for neutralizing said oxidized products. The methods include administering to a patient suffering from such a disease a composition comprising either deuterated arachidonic acid or a prodrug thereof. In some embodiments, these methods treat neurodegenerative diseases mediated by intracellular concentrations of 15-hydroperoxy-(Hp)-arachidonoyl-phophatidylethanolamine (15-HpETE-PE) by limiting the generation of this neurotoxin.

### CROSS-REFERENCE

This application claims priority to U.S. Provisional Application No. 63/240,751, filed Sep. 3, 2021, U.S. Provisional Application No. 63/253,061, filed Oct. 6, 2021, U.S. Provisional Application No. 63/253,690, filed Oct. 8, 2021, and U.S. Provisional Application No. 63/293,219, filed Dec. 23, 2021, each of which is incorporated by reference herein in its entirety.

### TECHNICAL FIELD

Disclosed are methods for inhibiting the progression of neurodegenerative diseases in humans. The methods may treat patients suffering from a neurodegenerative disease treatable with a deuterated arachidonic acid or a prodrug thereof. Disclosed are methods for inhibiting cellular dysfunctionality and subsequent cell death due to cellular accumulation of oxidized polyunsaturated fatty acids (PUFAs) products. Such cellular accumulation of oxidized PUFA products is due, at least in part, to the inability of regulatory enzymes to neutralize these oxidized products. In some embodiments, the subsequent cell death occurs via a regulatory cell death pathway such as apoptosis.

### BACKGROUND

There are a number of debilitating neurodegenerative diseases in humans which, despite the best efforts of researchers, remain incurable and often fatal. As such, the best the attending clinician can do is to slow the progression of the disease and, where possible, maintain a meaningful quality of life for the patient for as long as possible. Examples of such neurodegenerative diseases include the following:
amyotrophic lateral sclerosis (ALS) which is a late-onset, progressive neurological disease with its corresponding pathological hallmarks including progressive muscle weakness, muscle atrophy and spasticity all of which reflect the degeneration and death of upper and/or lower motor neurons. Once diagnosed, most patients undergo a rapid rate of disease progression terminating in death typically within 3 to 4 years with some patients succumbing even earlier;
tauopathy is a subgroup of Lewy body diseases or proteinopathies and comprises neurodegenerative conditions involving the aggregation of tau protein into insoluble tangles. These aggregates/tangles form from hyperphosphorylation of tau protein in the human brain. Specific conditions related to tauopathy include, but are not limited to, argyrophilic grain disease (AGD), chronic traumatic encephalopathy (CTE), corticobasal degeneration (CBD), frontotemporal dementia and parkinsonism linked to chromosome 17 (FTDP-17), ganglioglioma, gangliocytoma, lipofuscinosis, lytico-bodig disease, meningioangiomatosis, pantothenate kinase-associated neurodegeneration (PKAN), Pick's disease, postencephalitic parkinsonism, primary age-related tauopathy (PART), Steele-Richardson-Olszewski syndrome (SROS), and subacute sclerosing panencephalitis (SSPE). Wang et al., Nature Rev. Neurosci. 2016; 17:5 and Arendt et al., Brain Res. Bulletin 2016; 126:238. Tauopathies often overlap with synucleinopathies.
Steele-Richardson-Olszewski syndrome or progressive supranuclear palsy (PSP) is one example of a neurodegenerative disease mediated at least in part by tauopathy and involves the gradual deterioration and death of specific volumes of the brain. The condition leads to symptoms including loss of balance, slowing of movement, difficulty moving the eyes, and dementia. A variant in the gene for tau protein called the H1 haplotype, located on chromosome 17, has been linked to PSP. Besides tauopathy, mitochondrial dysfunction seems to be a factor involved in PSP. Especially, mitochondrial complex I inhibitors are implicated in PSP-like brain injuries;
Friedreich's ataxia is an autosomal-recessive genetic disease that causes difficulty walking, a loss of sensation in the arms and legs, and impaired speech that worsens over time. The pathology of this neurodegenerative disease involves degeneration of nerve tissue in the spinal cord;
Huntington's disease is a fatal genetic disorder that causes the progressive breakdown of nerve cells in the brain;
Corticobasal disorder (CBD) is a rare neurodegenerative disease characterized by
gradual worsening problems with movement, speech, memory and swallowing. It's often also called corticobasal syndrome (CBS). CBD is caused by increasing numbers of brain cells becoming damaged or dying over time;
Frontotemporal dementia (FTD) is a neurodegenerative disease and a common cause of dementia. It is characterized by a group of disorders that occur when nerve cells in the frontal temporal lobes of the brain are lost thereby causing the lobes to shrink. FTD can affect behavior, personality, language, and movement;
Nonfluent variant primary progressive aphasia (nfvPPA) occurs as a result of a buildup of one of two proteins, either tau or TPD-43, usually in the front left part of the brain. That part of the brain controls speech and language. As more of the protein builds up in those brain cells, the cells lose their ability to function and eventually die. As more cells die, the affected portion of the brain shrinks; and
late onset Tay-Sachs is a very rare genetic neurodegenerative disease in which fatty compounds, called gangliosides, do not break down fully because the body produces too little of the enzyme hexosaminidase A (or hex A). Over time, gangliosides build up in the brain and damage brain nerve cells. This affects a person's mental functioning.

There remains a need for treatments for these and other neurodegenerative diseases.

### SUMMARY

Generally, disclosed are methods for inhibiting cellular dysfunctionality and subsequent cell death due to cellular accumulation of oxidized PUFA products. These methods compensate for the reduced ability of regulatory enzymes to neutralize the oxidized PUFA products either due to genetic errors, disease progression, and/or age. As the oxidized PUFA products accumulate in the cell, cellular dysfunction occurs. If the accumulation is left unabated, the cell dies.

In some embodiments, the disclosed methods comprise incorporating deuterated arachidonic acid into said cell and components thereof in sufficient amounts to limit the amount of oxidized arachidonic acid generated to a level that said impaired enzymatic processes are capable of neutralizing substantially all of said oxidized products. Both the reduction in the amount of oxidized PUFA products generated and the ability of regulatory enzymes to neutralize the reduced amount of oxidized products still generated are evidenced by a substantial reduction in the rate of disease progression in the treated patient.

In some embodiments, there is provided a method for inhibiting cellular dysfunctionality and subsequent cell death due directly or indirectly to cellular accumulation of oxidized PUFA products as a result of impaired enzymatic process(es) that limit the neutralization of said oxidized products, said method comprises incorporating deuterated arachidonic acid into said cell and components thereof in sufficient amounts to reduce the amount of oxidized PUFAs generated to a level that said impaired enzymatic processes are capable of neutralizing substantially all of said oxidized products produced thereby inhibiting cellular dysfunctionality and subsequent cell death. Cells include any and all cells, for example any cell or cell type in a mammal, e.g., a human.

In some embodiments, the impairment responsible for reducing the ability of regulatory enzymes responsible for neutralizing oxidized PUFA products may be due to genetic defects leading to impaired enzymes with limited activity. The impairment may be a reduction of the amount of enzyme expressed or a reduction in the activity of the enzyme. The impairment may be age related or due to age. The impairment may also be due to age related limitations on the amount of enzyme expressed and/or a reduction in the activity of the enzymes so expressed. The impairment may also be due to the inability of the cell to produce sufficient enzyme to counter an increasing amount of oxidized PUFA products arising from a diseased condition. Finally, the impairment may be due to a combination of two or more of these factors.

In some embodiments, there is provided a method for restoring at least a portion of cellular functionality lost in dysfunctional cells which method comprises incorporating deuterated arachidonic acid into said cell and components thereof in sufficient amounts to limit the amount of oxidized PUFA products generated to a level that said impaired enzymatic processes are capable of neutralizing substantially all of said oxidized products thereby revitalizing said cell and, upon revitalization, restoring at least a portion of the functionality lost by said cells.

In some embodiments, there is provided a method to treat a neurodegenerative disease in a patient wherein said disease is mediated, directly or indirectly, by neural accumulation of oxidized PUFA products due to the failure of existing regulatory enzymes to neutralize all or substantially all of said products, said method comprises administering a sufficient amount of a deuterated arachidonic acid or a prodrug thereof, to said patient for a sufficient period of time such that the concentration of said deuterated arachidonic acid in red blood cells stabilizes the cells against oxidative processes and reduces the amount of oxidized PUFA products generated to a level that the existing regulatory enzymatic processes are capable of neutralizing more or most all of said oxidized products thereby treating said disease

In some embodiments, there is provided a method to treat a neurodegenerative disease in a patient wherein said disease is mediated, directly or indirectly, by neural accumulation of oxidized PUFA products as a result of impaired enzymatic process(es) that limit the amount of said oxidized products that can be neutralized, said method comprises administering a sufficient amount of deuterated arachidonic acid to said patient for a sufficient period of time such that the concentration of said deuterated arachidonic acid in red blood cells ranges from about 12% to about 25% based on the total amount of arachidonic acid including deuterated arachidonic acid thereby limiting the amount of oxidized PUFA products generated to a level that said impaired enzymatic processes are capable of neutralizing substantially all of said oxidized products thereby treating said disease.

In some embodiments, there is provided a method to treat a neurodegenerative disease in a patient wherein said disease is mediated by neural accumulation of oxidized PUFA products as a result of impaired enzymatic process(es) that limit the amount of said oxidized products that can be neutralized, said method comprises administering a sufficient amount of 11,11-D2-linoleic acid to said patient for a sufficient period of time such that a concentration of 13,13-D2-arachidonic acid in red blood cells ranges from about 12% to about 25% based on the total amount of arachidonic acid including deuterated arachidonic acid, thereby limiting the amount of oxidized PUFA products generated to a level that said impaired enzymatic process(es) are capable of neutralizing substantially all of said oxidized products, thereby treating said disease. In some embodiments, a concentration of 13,13-D2-arachidonic acid in red blood cells in a blood sample obtained from said patient was assessed. In some embodiments, a concentration of 13,13-D2-arachadonic acid was obtained at a set period after start of therapy and compared to a control. In some embodiments, a control is a standardized concentration curve. In some embodiments, the method further comprising assessing whether the amount of 11,11-D2-linoleic acid or ester thereof administered to the patient should be changed. In some embodiment, the amount of 11,11-D2-linoleic acid or ester thereof administered to the patient should be increased if the concentration of 13,13-D2-arachidonic acid in the red blood cells is lower than the control.

In some embodiments, cell death is the result of a regulatory cell death pathway. In another embodiment, the regulatory cell death pathway is selected from the group consisting of intrinsic apoptosis, extrinsic apoptosis, mitochondrial permeability transition (MPT)-driven necrosis, necroptosis, oxytosis, ferroptosis, and pyroptosis. In some embodiments, wherein the cell death in initiated by the presence of a sufficient amount of 15-HpETE-PE to trigger the cellular death signal.

In some embodiments, the cell is a neuron.

In another aspect, disclosed are methods for treating patients diagnosed with a neurodegenerative disease wherein neuronal cell death is mediated at least in part by intraneuronal concentrations of 15-HpETE-PE neurotoxin that trigger a death signal in those neurons thereby accounting for the progression of the disease. Patients so diagnosed are treated over a prolonged period of time with sufficient amounts of deuterated arachidonic acid or a prodrug thereof. The deuterated arachidonic acid accumulates over time into at-risk neurons of a patient until these cells are able to maintain the concentration of 15-HpETE-PE below that which triggers the death signal.

Without being limited to any theory, those neurons containing sufficient deuterated arachidonic acid with a sufficiently long half-life to resist oxidation at the bis-allylic sites of deuterated arachidonic acid by reactive oxygen species (ROS) as compared to wild type arachidonic acid. This resistance imparts enhanced stability to the neurons which reduces the amount of 15-peroxidized arachidonic acid in the phospholipids, a precursor of 15-HpETE-PE. This, in turn, limits the amount of 15-HpETE-PE present in a neuron by minimizing the amount of these toxic byproducts of lipid peroxidation (LPO) thus controlling the level of 15-HpETE-PE to a level that is below that necessary to trigger the death signal. Controlling the amount of 15-HpETE-PE generated limits the extent of programed cell death in neurons responsible for vital functions in the patient leading to prolongation of life and vital functionality.

In some embodiments, there is provided a method for inhibiting at risk cells from generating a concentration of 15-HpETE-PE that signals for cell death and constitutes a death signal, comprising a) contacting a population of at-risk cells with an effective amount of a deuterated arachidonic acid or a prodrug thereof, under conditions wherein said deuterated arachidonic acid is incorporated into the cells and components thereof; b) maintaining said contact thereof under conditions wherein the increase in the intracellular concentration of 15-HpETE-PE, is reduced or eliminated thereby delaying or preventing cellular death due to triggering of the 15-HpETE-PE cellular death signal.

In another embodiment, there is provided a method for treating a patient diagnosed with a neurodegenerative disease mediated, at least in part, by 15-HpETE-PE, wherein said 15-HpETE-PE, at a sufficient concentration, signals for neuronal death and constitutes a death signal, said method comprises administering to said patient an effective amount of a deuterated arachidonic acid or a prodrug thereof under conditions wherein the increase in the intracellular concentration of 15-HpETE-PE, is reduced or eliminated thereby delaying or preventing cellular death due to triggering of the 15-HpETE-PE cellular death.

In many cases, late-stage patients with neurodegenerative diseases succumb to the disease due to a loss of neurons that control one or more vital functionalities such as swallowing and/or breathing. In the case of swallowing, the loss of muscle control in the mouth, the tongue, the larynx, the pharynx, control of the airway and maintaining breathing thru the nose while swallowing or drinking and/or the esophagus can lead to aspiration into the lungs of food, water, and the like. Aspiration of such materials into the lungs leads to a significant risk of pneumonia and death.

In some embodiments, there is provided a method for prolonging vital functionality in a patient suffering from a neurodegenerative disease targeting the neurons responsible for maintaining vital functionality wherein said disease is mediated, at least in part by neurotoxin 15-HpETE-PE, wherein said neurotoxin, at a sufficient concentration, signals for neuronal death and constitutes a death signal for at-risk neurons, said method comprises: administering to said patient an effective amount of a deuterated arachidonic acid or a prodrug thereof; and maintaining said administration over a period of time sufficient to reduce the concentration of peroxidized arachidonic acid in phospholipids (including lysophospholipid) of at-risk neurons wherein said phospholipids comprise 15-HpETE—wherein said limit in the increase in the concentration of or reduction in the concentration of 15-HpETE-PE inhibits initiation of the death signal thereby prolonging the vital functionality of said patient.

It is understood that by maintaining vital functionality in such patients, deaths associated with loss of such functionality are delayed thereby extending the life of such patients.

1. In some embodiments, the deuterated arachidonic acid is oxidated at the 13-position to generate 15-HPAP. In some embodiments, the deuterated arachidonic acid is a D6-arachidonic acid characterized as a composition of deuterated arachidonic acid or a prodrug thereof that comprises, on average, at least about 80% of the hydrogen atoms at each of the bis-allylic sites having been replaced by deuterium atoms and, on average, no more than about 35% of the hydrogen atoms at the mono-allylic sites having been replaced by deuterium atoms. In some embodiments, deuterated arachidonic acid or prodrug thereof is administered to the patient such that a concentration of the deuterated arachidonic acid in red blood cells reaches at least about 12% based on the total amount of arachidonic acid in the red blood cells including the deuterated arachidonic acid and preferably at least 20%. In some embodiments, the amount of deuterated arachidonic acid in red blood cells ranges from about 10% to about 30% and, preferably from about 12% to about 25% at six months after initiation of treatment.

In some embodiments, the generation of 15-HpETE-PE includes oxidation at the 13-position of arachidonic acid which is a bis-allylic site in the phospholipids found in the neurons. As a result, the deuterated arachidonic acid employed has at least one deuterium replacing hydrogen and preferably, both hydrogens at this position are replaced by deuterium.

In some embodiments, the deuterated arachidonic acid is 13,13-D2-arachidonic acid.

In some embodiments, the deuterated arachidonic acid is 10,10,13,13-D4-arachidonic acid.

In some embodiments, the deuterated arachidonic acid is 7,7,10,10,13,13-D6-arachidonic acid.

In some embodiments, the deuterated arachidonic acid is a D6-arachidonic acid characterized as a composition of deuterated arachidonic acid or a prodrug thereof that comprises, on average, at least about 80% of the hydrogen atoms at each of the bis-allylic sites having been replaced by deuterium atoms and, on average, no more than about 35% of the hydrogen atoms at the mono-allylic sites having been replaced by deuterium atoms.

In some embodiments, the deuterated PUFA administered to the patient is 11,11-D2-linoleic acid or an ester thereof which acts as a prodrug for 13,13-D2-arachidonic acid.

In some embodiments, the deuterated PUFA administered to the patient is 13,13-D2-arachidonic acid or a prodrug thereof. The prodrug may be an ester of 13,13-D2-arachidonic acid that is rapidly deesterified after ingestion.

In some embodiments, the deuterated PUFA administered to the patient is 7,7,10,10,13,13-D2-arachidonic acid or a prodrug thereof. The prodrug may be an ester of 7,7,10,10,13,13-D2-arachidonic acid.

In some embodiments, sufficient deuterated arachidonic acid is administered to the patient such that a concentration of the deuterated arachidonic acid in red blood cells reaches at least about 12% based on the total amount of arachidonic acid in the red blood cells including the deuterated arachidonic acid.

In some embodiments, the amount of deuterated arachidonic acid in red blood cells ranges from about 12% to about 25% at six months after initiation of treatment. In some embodiments, the amount of deuterated arachidonic acid in red blood cells ranges up to 50% or more based on the total amount of arachidonic acid present including deuterated arachidonic acid.

In another aspect, methods are disclosed that significantly attenuate the progression of neurodegenerative diseases treatable by administration of deuterated arachidonic acid or a prodrug thereof. Such administration is delivered with a dosing regimen that comprises both a loading regimen and a maintenance regimen. The loading regimen ensures that there is a rapid onset to therapeutic levels of the deuterated arachidonic acid in vivo to attenuate disease progression. This results in the retention of more functionality in the patient as compared to dosing regimens that require longer periods of time to achieve therapeutic levels. The maintenance dose ensures that the therapeutic levels of the deuterated arachidonic acid are maintained in the patient during therapy.

In some embodiments, the deuterated arachidonic acid or a prodrug thereof has one or more deuterium atoms at the bis-allylic sites. In some embodiments, the deuterated arachidonic acid or a prodrug thereof is 13,13-D2-arachidonic acid or a prodrug thereof, 10,10,13,13-D4-arachidonic acid or a prodrug thereof, or 7,7,10,10,13,13-D2-arachidonic acid or a prodrug thereof. In another embodiment, there is provided a composition of deuterated arachidonic acid or a prodrug thereof which composition comprises on average at least about 80% of the hydrogen atoms at the bis-allylic sites replaced by deuterium atoms. In some embodiments, the deuterated arachidonic acid or a prodrug thereof comprises on average at least about 80% of the hydrogen atoms at the bis-allylic sites replaced by deuterium atoms and no more than about 35% on average of the hydrogen atoms at the mono-allylic sites replaced by deuterium atoms.

In some embodiments, the deuterated arachidonic acid or a prodrug thereof is 13,13-D2-arachidonic acid or a prodrug thereof.

In some embodiments, the deuterated arachidonic acid or a prodrug thereof is 10,10,13,13-D4-arachidonic acid or a prodrug thereof.

In some embodiments, the deuterated arachidonic acid or a prodrug thereof is 7,7,10,10,13,13-D6-arachidonic acid or a prodrug thereof.

Without being limited by theory, once administered, deuterated arachidonic acid is systemically absorbed and incorporated into cells, such as the cell membrane and the mitochondria. In neurons, the deuterated arachidonic acid stabilizes the cell membrane against oxidative damage caused by reactive oxygen species. This, in turn, stops the cascade of lipid peroxidation, thereby minimizing damage to motor neurons where the deuterated arachidonic acid is incorporated. When concentrations of deuterated arachidonic acid reach a therapeutic level in the motor neurons, the disease progression of neurodegenerative diseases is significantly attenuated.

The methods described herein provide for rapid onset of a therapeutic concentration of deuterated arachidonic acid in vivo so as to minimize unnecessary loss of functionality in the treated patients suffering from a neurodegenerative disease. In some embodiments, there is provided a method for reducing disease progression of a neurodegenerative disease in an adult patient treatable with deuterated arachidonic acid while providing for rapid onset of therapy, the method comprising periodically administering deuterated arachidonic acid or a prodrug thereof to the patient with a dosing regimen that comprises a primer dose and a maintenance dose.

In an embodiment, the primer dose comprises periodic administration of deuterated arachidonic acid or a prodrug thereof. In an embodiment, the primer dose comprises at least about 10 milligrams of deuterated arachidonic acid or a prodrug thereof per day. In an embodiment, the primer dose comprises from about 50 milligrams to about 2 grams of deuterated arachidonic acid or a prodrug thereof per day. In an embodiment, the primer dose comprises from about 0.10 grams to about 1 gram. In an embodiment, the primer dose is continued for about 15 to about 50 days or from about 30 days to about 45 days, e.g., to rapidly achieve a therapeutic concentration of deuterated arachidonic acid in vivo, thereby reducing the rate of disease progression.

In an embodiment, after completion of the primer dose, the maintenance dose is periodically administered. In an embodiment, no more than about 65% of the loading dose of the deuterated arachidonic acid or a prodrug thereof per day is administered as a maintenance dose. In an embodiment, the maintenance dose is utilized to ensure that the therapeutic concentration of deuterated arachidonic acid is maintained in vivo such that a reduced rate of disease progression is maintained.

In an embodiment, the reduced rate of disease progression is evaluated when compared to the rate of disease progression measured prior to initiation of said method. In an embodiment, each of said neurodegenerative diseases is mediated at least in part by lipid peroxidation of polyunsaturated fatty acids in neurons of the patient suffering from said neurodegenerative disease.

In some embodiments, said neurodegenerative disease is amyotrophic lateral sclerosis (ALS), Huntington's Disease, progressive supernuclear palsy (PSP), APO-e4 Alzheimer's Disease, corticobasal disorder (CBD), frontotemporal dementia (FTD), nonfluent variant primary progressive aphasia (nfvPPA), other tauopathies, or late onset Tay-Sachs.

In some embodiments, said periodic administration of the loading dose comprises administration of from about 0.05 grams to about 2 grams of deuterated arachidonic acid or a prodrug thereof per day. In embodiments, the loading dose is administered for at least 5 days per week, and preferably 7 days a week.

In some embodiments, the periodic administration of the maintenance dose of deuterated arachidonic acid or a prodrug thereof per day comprises no more than 55% of the loading dose. In embodiments, the maintenance dose is administered per day, or at least 5 days per week, or at least once per week, or at least once per month. In another embodiment, the maintenance dose comprises no more than 35% of the loading dose which is administered at least once a month.

In some embodiments, the periodic administration of the maintenance dose is calibrated to be an amount of deuterated arachidonic acid or a prodrug thereof sufficient to replace the amount of deuterated arachidonic acid eliminated from the body.

In some embodiments, the percent reduction in the rate of disease progression is determined by:

measuring a natural rate of disease progression in a patient or an average natural rate of disease progression in a cohort of patients prior to initiation of therapy per the methods described herein;

measuring the rate of disease progression in said patient or cohort of patients during a period of compliance with the periodic administration of both the loading dose and the maintenance dose; and

after said period of compliance from the start of therapy, optionally annualizing the progression rate during the natural history and the progression rate during therapy, calculating the difference between the natural rate and the rate during the period of compliance, dividing the difference by the rate of disease progression during the natural history of the patient, and multiplying by 100.

In some embodiments, the set period of time is between about 1 month and about 24 months, for example about 3 months, about 6 months or about 12 months, or about 18 months or about 24 months. In an embodiment, the set period of time is at least 3 months.

In some embodiments, the methods described herein further comprise restricting the patient's consumption of excessive dietary polyunsaturated fatty acids during administration of said primer and said maintenance doses.

In some embodiments, there is provided a kit of parts comprising a set of capsules, each capsule comprising a partial loading dose of deuterated arachidonic acid or a prodrug thereof, such that two or more of said capsules comprise a complete loading dose per day.

In some embodiments, there is provided a kit of parts comprising a set of capsules, each capsule comprising a partial loading dose of deuterated arachidonic acid or a prodrug thereof, such that no more than four of said capsules comprise a complete loading dose per day.

In some embodiments, there is provided a kit of parts comprising a set of capsules, each capsule comprising a partial maintenance dose of deuterated arachidonic acid or a prodrug thereof, such that two or more of said capsules comprise a complete maintenance dose per day.

In some embodiments, there is provided a kit of parts comprising a set of capsules, each capsule comprising a partial maintenance dose of deuterated arachidonic acid or a prodrug thereof such that one or two of said capsules comprise a complete maintenance dose per day.

In some embodiments, the percent reduction in the rate of disease progression from that occurring during the natural history of the patient and after start of therapy is at least 25%, at least 30%, preferably at least 40%, more preferably at least 65% and most preferably greater than 70% or 80% after 3 or 6 months. Accordingly, in some embodiments, methods disclosed herein provide for determining a percent reduction in the rate of disease progression by (i) determining a natural rate of disease progression in a patient or an average natural rate of disease progression in a cohort of patients; (ii) determining the rate of disease progression in the patient or cohort of patients during a period of compliance with administration of deuterated arachidonic acid, or a prodrug thereof; (iii) measuring the difference between the natural rate of disease progression and the rate during the period of compliance, (iv) optionally annualizing the progression rate during the natural history and the progression rate during therapy; (v) dividing the difference by the natural rate of disease progression and (vi) multiplying by 100.

In some embodiments, whether a therapeutic concentration of deuterated arachidonic acid has been reached in neurons is measured using a reporter cell. In an embodiment, the reporter cells are red blood cells. In the case of red blood cells, using 13,13-D2-arachidonic acid as an example, a concentration of 13,13-D2-arachidonic acid of at least about 3% based on the total number of arachidonic acid, including deuterated arachidonic acid, contained in the red blood cells has been found to correlate with therapeutic results. See, e.g., U.S. Provisional Patent Application No. 63/177,794, filed Apr. 21, 2021, which is incorporated by reference in its entirety.

In some embodiments, the patients are placed on a diet that restricts intake of excessive amounts of polyunsaturated fatty acids (PUFAs). This is because as more PUFAs are consumed by the patient, the percentage of deuterated arachidonic acid or a prodrug thereof is lowered since it is administered at a fixed dose. Since the clinician is attempting to increase the percentage of deuterated arachidonic acid in vivo, lowering the percentage of that drug consumed relative to the total amount of arachidonic acid consumed is counter intuitive. Generally, dietary components that contribute to excessive amounts of PUFA consumed are restricted including, for example, fish oil pills, products that contain high levels of PUFAs, such as salmon; patients on conventional feeding tubes may also have excessive PUFA intake. In a preferred embodiment, the methods described herein include both the dosing regimen described above as well as placing the patients on a restrictive diet that avoids excessive ingestion of PUFA components and especially excessive linoleic acid.

In some embodiments, there is provided a method for reducing the rate of disease progression in a patient suffering from a neurodegenerative disease treatable with deuterated arachidonic acid, which method comprises administering deuterated arachidonic acid or a prodrug thereof to the patient with a dosing regimen that comprises a primer dosing and a maintenance dosing schedule which comprise:

a) said first dosing component comprises administering to said patient a primer dose of deuterated arachidonic acid or a prodrug thereof in an amount and for a period of time sufficient to allow for reduction in the rate of disease progression from start of dosing;

b) subsequently following said primer dose, initiating a maintenance dose to said patient, said maintenance dose comprises an amount of deuterated arachidonic acid or a prodrug thereof in an amount sufficient to maintain the concentration of deuterated arachidonic acid in the motor neurons, wherein the amount of deuterated arachidonic acid or a prodrug thereof administered in said maintenance dose is less than the amount administered in said primer dose; and optionally:

c) monitoring the concentration of deuterated arachidonic acid in the patient to ensure that the patient is maintaining a therapeutic concentration; and

d) increasing the dosing of deuterated arachidonic acid or a prodrug thereof when said concentration is deemed to be less than a therapeutic amount.

### BRIEF DESCRIPTION OF THE DRAWINGS

FIG. 1 is a scatterplot and standard fit showing the percent of 13,13-D2-Arachidonic Acid in red blood cells (RBC) and cerebral spinal fluid (CSF) over time after treatment with 11,11-D2-Linoleic Acid in an adult patient.

FIG. 2 is a scatterplot and standard fit showing the percent of 13,13-D2-Arachidonic Acid in red blood cells (RBC) and cerebral spinal fluid (CSF) over time after treatment with 11,11-D2-Linoleic Acid in juvenile patients.

### DETAILED DESCRIPTION

This disclosure is directed to methods for treating neurodegenerative diseases by administrating of deuterated arachidonic acid or a prodrug thereof. In some embodiments, the methods comprise inhibiting cellular dysfunctionality and subsequent cell death due to cellular accumulation of oxidized arachidonic acid products as a result of impaired enzymatic process(es) that limit the neutralization of said oxidized products. In some embodiments, the methods comprise treating neurodegenerative diseases mediated by neuronal death due to toxic intracellular concentrations of 15-HpETE-PE by limiting the generation of this neurotoxin. In some embodiments, the methods of this disclosure include a dosing regimen that is sufficient to provide a therapeutic level of deuterated arachidonic acid in the motor neurons. In another embodiment, the methods described herein comprise a daily or periodic primer or loading dose that accelerates delivery of deuterated arachidonic acid to the diseased neurons of the patient. This primer dose is continued for a sufficient period of time to achieve a therapeutic concentration of a deuterated arachidonic acid in vivo. At that point, a daily or periodic maintenance dose is employed to maintain the therapeutic concentration of the deuterated arachidonic acid.

Prior to discussing this invention in more detail, the following terms will first be defined. Terms that are not defined are given their definition in context or are given their medically acceptable definition.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by (+) or (−) 15%, 10%, 5%, 1%, or any subrange or subvalue there between. Preferably, the term “about” when used with regard to a dose amount means that the dose may vary by +/−10%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the term “consisting of” shall mean excluding more than trace elements of other ingredients and substantial method steps. Embodiments defined by each of these transition terms are within the scope of this invention.

As used herein, arachidonic acid has the numbering system as described below:

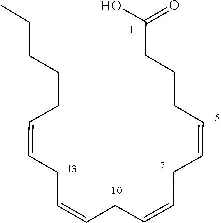 [SMILES: CCCCC/C=C\C/C=C\C/C=C\C/C=C\CCCC(=O)O]

where each of positions 7, 10 and 13 are bis-allylic positions within the structure.

As used herein and unless the context dictates otherwise, the term “deuterated arachidonic acid or a prodrug thereof” refers to arachidonic acid as well as esters thereof having deuteration as described below. In vivo, esters are first hydrolyzed to provide for the corresponding acid or salt thereof and then incorporated into structural features such as glycerol esters. A “deuterated arachidonic acid or a prodrug thereof” may be a 7-D1-arachidonic acid or a prodrug thereof; 10-D1-arachidonic acid or a prodrug thereof; 13-D1-arachidonic acid or a prodrug thereof; 7,10-D2-arachidonic acid or a prodrug thereof; 7,13-D2-arachidonic acid or a prodrug thereof; 10,13-D2-arachidonic acid or a prodrug thereof; 7,7-D2-arachidonic acid or a prodrug thereof; 10,10-D2-arachidonic acid or a prodrug thereof; 13,13-D2-arachidonic acid or a prodrug thereof; 7,10,13-D3-arachidonic acid or a prodrug thereof; 7,7,10-D3-arachidonic acid or a prodrug thereof; 7,10,10-D3-arachidonic acid or a prodrug thereof; 7,13,13-D3-arachidonic acid or a prodrug thereof; 10,10,13-D3-arachidonic acid or a prodrug thereof; 10,13,13-D3-arachidonic acid or a prodrug thereof; 7,7,10,13-D4-arachidonic acid or a prodrug thereof; 7,7,10,10-D4-arachidonic acid or a prodrug thereof; 7,10,10,13-D4-arachidonic acid or a prodrug thereof; 7,10,13,13-D4-arachidonic acid or a prodrug thereof; 7,7,13,13-D4-arachidonic acid or a prodrug thereof; 10,10,13,13-D4-arachidonic acid or a prodrug thereof; 7,7,10,10,13-D5-arachidonic acid or a prodrug thereof; 7,7,10,13,13-D5-arachidonic acid or a prodrug thereof; 7,10,10,13,13-D5-arachidonic acid or a prodrug thereof 7,7,10,10,13,13-D6-arachidonic acid or a prodrug thereof; or mixtures of any two or more.

D2-arachidonic acids include 7,7-D2-arachidonic acid or prodrugs thereof; 10,10-D2-arachidonic acid or prodrugs thereof and 13,13-D2-arachidonic acid or prodrugs thereof.

D4-arachidonic acids or prodrugs thereof include 7,7,10,10-D4-arachidonic acid or prodrugs thereof 7,7,13,13-D4-arachidonic acid or prodrugs thereof and 10,10,13,13-D4-arachidonic acid or prodrugs thereof. In some embodiments, 10,10,13,13-D4-arachidonic acid can be biosynthesized from 8,8,11,11-D4-gamma linolenic acid or from 10,10,13,13-D6-d-homa-gamma linolenic acid. The bioconversion of both of these PUFAs results in 10,10,13,13-D4-arachidonic acid. Both the 8,8,11,11-D4-gamma linolenic acid or the 10,10,13,13-D6-d-homa-gamma linolenic acid (or esters of either) can be prepared by ruthenium catalysis as described below provided that such will result in at least 80% deuteration of their bis-allylic positions as well as nominal amounts of deuteration at one or both of the mono-allylic positions (e.g., less than about 25%).

D6-arachidonic acid includes 7,7,10,10,13,13-D6-arachidonic acid or prodrugs thereof.

As to deuteration, such is described as an average based on a population of such compounds comprising a total deuteration refers to 7,7,10,10,13,13-D6-arachidonic acid or a prodrug thereof including compositions of deuterated arachidonic acid or a prodrug thereof that comprises, on average, at least about 80% of the hydrogen atoms at each of the bis-allylic sites having been replaced by deuterium atoms and, on average, no more than about 35% of the hydrogen atoms at the mono-allylic sites having been replaced by deuterium atoms. For example, in the case of 80% deuteration of the 3 bis-allylic sites and 35% deuteration of the mono-allylic sites, the total amount of deuterium is (6×0.8)+(4×0.35)=6.2 exclusive of the naturally occurring amount of deuterium in each of the remaining methylene and methyl groups within the structure.

In some embodiments, the amount of deuterium replacing hydrogen at the bis-allylic sites (7, 10 and 13) and at the mono-allylic sites (4 and 16) of arachidonic acid can be any one of the following: at least about 85% deuterium at bis-allylic sites/no more than about 30% at mono-allylic sites; at least about 85% deuterium at bis-allylic sites/no more than about 25% at mono-allylic sites; at least about 85% deuterium at bis-allylic sites/no more than about 20% at mono-allylic sites; at least about 85% deuterium at bis-allylic sites/no more than about 10% at mono-allylic sites; at least about 85% deuterium at bis-allylic sites/no more than about 5% at mono-allylic sites; at least about 90% deuterium at bis-allylic sites/no more than about 30% at mono-allylic sites; at least about 90% deuterium at bis-allylic sites/no more than about 25% at mono-allylic sites; at least about 90% deuterium at bis-allylic sites/no more than about 20% at mono-allylic sites; at least about 90% deuterium at bis-allylic sites/no more than about 10% at mono-allylic sites; and at least about 90% deuterium at bis-allylic sites/no more than about 5% at mono-allylic sites.

The term “prodrug” as it relates to deuterated arachidonic acid includes esters of arachidonic acid (as defined below) as well as 11,11-linoleic acid or esters thereof. As to the esters, oral administration of esters of arachidonic acid or linoleic acid will result in deesterification in the gastro-intestinal tract thereby generating arachidonic acid or linoleic acid. As to the latter, a portion of 11,11-D2-linoleic acid is bioconverted into 13,13-D2-arachidonic acid and therefore this compound acts as a prodrug of 13,13-arachidonic acid.

As used herein and unless the context dictates otherwise, the term “an ester thereof” refers to a C₁-C₆ alkyl esters, glycerol esters (including monoglycerides, diglycerides and triglycerides), sucrose esters, phosphate esters, and the like. The particular ester group employed is not critical provided that the ester is pharmaceutically acceptable (non-toxic and biocompatible). In some embodiments, the ester is a C₁-C₆ alkyl ester which is preferably an ethyl ester.

As used herein, the term “phospholipid” refers to any and all phospholipids that are components of the cell membrane. Included within this term are phosphatidylcholine, phosphatidylethanolamine, phosphatidylserine, and sphingomyelin. In the motor neurons, the cell membrane is enriched in phospholipids comprising arachidonic acid.

The term “bis-allylic site” refers to the methylene group (CH₂) separating two double bonds.

The term mono-allylic site” refers to the methylene group have an adjacent neighboring double bond on one side and a further methylene group on the opposite side.

The term “cellular component” refers to any intracellular structure found in human cells including organelles having a lipid or phospholipid wall. Examples of such cellular components include by way of example only the mitochondria, the endoplasmic reticulum, golgi apparatus, and the like.

The term “regulatory enzymes” as it relates to the neutralization of oxidized arachidonic acid products refer to those enzymes that are responsible to remove, alter, or destroy one or more of the oxidized arachidonic acid products from the cells to prevent the accumulation of these oxidized products within the cell.

The term “oxidized PUFA products” refer to any oxidized form of a polyunsaturated fatty acid as well as any and all metabolites formed from the oxidized PUFA including reactive aldehydes, ketones, alcohols, carboxyl derivatives which are toxic to the cell when found in a phospholipid, a lipid bilayer, or as an enzyme substrate.

The term “cellular dysfunctionality” refers to a cell's inability to properly function in conjunction with a population of similar cells that control essential functions of the body such as memory, motor skills, vital functionality such as breathing, swallowing, and the like. As more and more cells within this population exhibit cellular dysfunctionality, the overall dysfunctionality manifests itself in diminished to lost capacity for such functions. Loss of motor skills/functions are symptoms of ALS, ataxia, Huntington's Disease, neuropathy, to name a few. Loss of memory function is symptomatic of AD and other forms of dementia.

The term “impairment” as it relates to enzymes and enzymatic processes refers to the inability of one or more enzymes responsible for neutralizing oxidized arachidonic acid products to adequately prevent accumulation of said oxidized products. For example, the impairment may be due to genetic defects leading to impaired enzymes with limited activity. The impairment may also be due to age related limitations on the amount of enzyme expressed and/or a reduction in the activity of the enzymes so expressed. The impairment may also be due to the inability of the cell to produce sufficient enzyme to counter an increasing amount of oxidized arachidonic acid products arising from a diseased condition. Finally, the impairment may be due to a combination of one or more of these factors.

The term “neutralization of said oxidized products” refers to enzymatic processes that remove, alter, or destroy oxidized PUFA products from the cells to prevent the accumulation of these oxidized products within the cell. In healthy individuals, the neutralization of the oxidized PUFA products prevents accumulation of these products thereby assuring that dysfunctionality of cells especially neurodegenerative cells is avoided or controlled.

The term “restoring at least a portion of cellular functionality” refers to an improvement in one or more functional features of a patient, and, preferably, in one or more vital functionalities such as swallowing. In the case of swallowing, the restoration can be measured by the rate of aspiration of treated a patient or a cohort of patients before initiation of therapy versus after initiation of therapy. A reduction in the rate of aspiration of at least about 10% after the start of therapy as compared to before the start of therapy evidence restoration of some swallowing functionality. Preferably, the rate of reduction in aspiration is at least 20% or more preferably at least 25%.

As used herein, the term “pathology of a disease” refers to the cause, development, structural/functional changes, and natural history associated with that disease. The term “natural history” means the progression of the disease in the absence of treatment per the methods described herein.

The term “death signal” refers to an intraneuronal concentration of 15-HpETE-PE that signals for cell death by apoptosis, ferroptosis, or similarly related cellular processes that lead to cell death. Once initiated, the process is irreversible and contributes to the overall pathology of neurodegenerative diseases. Neuronal cell death in vital structures leads to further LPO and generation of yet more 15-HpETE-PE that overwhelms even a normal PLA2G6 based clearance system for this and other neurotoxins. This death signal is the ultimate endpoint for a biological cascade that includes the generation of 15-peroxidized phospholipids such as 15-peroxidized arachidonic acid as a component of such phospholipids. These oxidized phospholipids comprise oxidized arachidonic acid that starts with hydrogen extraction at the 13-bis-allylic position due to oxidative species such as ROS. Migration of the 14,15 double bond to the 13,14 carbon atoms and translocation of the oxide species provides for the 15-peroxidized arachidonic acid in these oxidized phospholipids.

The term “sufficient deuterated arachidonic acid” as it relates to that amount necessary to protect against generation of 15-peroxidized arachidonic acid refers to an amount of deuterated arachidonic acid incorporated into an at-risk neuron. When incorporated into the phospholipids such as PE of neurons, the ability of oxidizing agents such as Fe and LPO to initiate peroxidation at the 13-bis-allylic position of arachidonic acid in the phospholipids is reduced. In this manner, there is reduced production of 15-HpETE-PE in membranes of neurons in vital structures thereby reducing the triggering of the death signal.

As noted above, the generation of 15-HpETE-PE results from the generation of the 13-oxidized arachidonic acid in phospholipids. As such, the deuterated arachidonic acid compounds used in the methods described herein have at least one but preferably both hydrogen atoms at the 13-position of arachidonic acid replaced with deuterium. Such replacement stabilizes this position of arachidonic acid and the phospholipid containing it against oxidation as the carbon-deuterium bond is significantly more stable against oxidation than a carbon-hydrogen bond. This reduces metabolic formation of 15-HpETE-PE as well as other potential pathogenic metabolites.

As used herein, the term “at-risk neurons” refers to those neurons whose death is included in the pathology of the disease. For example, infants with INAD may have a set of neurons that are at-risk whereas Alzheimer's Disease may have other neurons that are at risk.

As used herein, the term “reduced rate of disease progression” means that the rate of disease progression is attenuated after initiation of treatment as compared to the patient's natural history. In one case, the rate of reduction in disease progression using the methods described herein results in a percentage reduction of at least 25% lower or at least 30% lower at a time point, e.g., 1 month to 24 months, e.g., 3 or 6 months, after initiation of therapy when compared to the natural history of the patient. In one case, the rate of reduction in disease progression using the methods described herein results in a percentage reduction of at least 75% lower or at least 90% lower at a time point either at 3 or 6 or 12 months after initiation of therapy when compared to the natural history of the patient. Such reduced rates of disease progression evidence that the level of enzymatic control of the oxidized PUFA products.

Alternatively, the reduction in the rate of disease progression is confirmed by a reduction in the downward slope (flattening the curve) of a patient's relative muscle functionality during therapy as compared to the downward slope found in the patient's natural history. Typically, the differential between the downward slope measured prior to treatment and the slope measured after at least 90 days from initiation of treatment has a flattening level of at least about 30%. So, a change of 7.5 degrees (e.g., a downward slope of 25 degrees during the natural history that is reduced to a downward slope of 17.5 degrees provides for a 40% decrease in the slope). In any case, the reduction in downward slope evidence that the patient has a reduced rate of disease progression due to the therapy.

The term “therapeutic concentration” means a concentration of a deuterated arachidonic acid that reduces the rate of disease progression by at least 25% or at least 30%. Since measuring the concentration of a deuterated arachidonic acid in the motor neurons or in the spinal fluid of a patient is either not feasible or optimal, the therapeutic concentration is based on the concentration of deuterated arachidonic acid found in red blood cells as provided in the Examples below. Accordingly, any reference made herein to a therapeutic concentration of deuterated arachidonic acid is made by evaluating its concentration in red blood cells.

In some embodiments, a concentration of 13,13-D2-arachidonic acid in red blood cells of from about 12 percent to about 25% based on the total amount of arachidonic acid found therein including deuterated arachidonic acid has been shown to be therapeutic at the levels set forth above. Preferably, a therapeutic concentration of 13,13-D2-arachidonic acid will range from about 15% to about 20% in red blood cells.

As used herein, the term “patient” refers to a human patient or a cohort of human patients suffering from a neurodegenerative disease treatable by administration of deuterated arachidonic acid or a prodrug thereof. The term “adult patient” refers to a subject over 18 years of age and suffering from a neurodegenerative disease treatable by administration of deuterated arachidonic acid or a prodrug thereof.

In some embodiments, only a fraction of the linoleic acid consumed per day is converted by the patient into arachidonic acid. The specific amount of arachidonic acid generated depends on the patient's physiology as well as the amount of PUFAs consumed per day. The higher the PUFA load consumed by the patient translates into a smaller amount of deuterated arachidonic acid generated in vivo.

As used herein, the term “loading or primer amount” of deuterated arachidonic acid or a prodrug thereof refers to an amount of a deuterated arachidonic acid or a prodrug thereof that is sufficient to provide for a reduced rate of disease progression within at least about 45 days after initiation of administration and preferably within 30 days. The amount so employed is loaded to accelerate the period of time to reduce the rate of disease progression within this time period. When less than a loading amount is used, it is understood that such can still provide for therapeutic results but the time period between start of therapy and when therapeutic results are achieved will be longer and, likely, will not achieve the same level of reduction in disease progression. Moreover, given the progressive nature of these neurodegenerative diseases, the use of the dosing regimens described herein will minimize the time necessary to achieve the desired reduction in the rate of disease progression thereby retaining as much of the patient's remaining muscle functionality while limiting further loss of functionality.

In some embodiments, the term “loading or primer amount” of deuterated linoleic acid or ester thereof refers to a sufficient amount of 11,11-D2-linoleic acid or an ester thereof that provides for in vivo conversion into therapeutic concentration of 13,13-D2-arachidonic acid. In some embodiments, the loading dose for an adult patient is about 9 grams per day of 11,11-D2-linoleic acid ethyl ester (e.g., about 8.55 grams when accounting for the ethyl ester which will be removed and a small percent of impurities) for at least 30 days. Afterwards, the duration of the loading dose is optionally continued or the dosing can be increased by the attending clinician depending on whether the analysis of deuterated arachidonic acid in the red blood cells evidence insufficient levels of deuterated red blood cells at about 30 days post start of therapy.

The methods described herein are based on the discovery that the primer doses of deuterated arachidonic acid or a prodrug thereof employed to date are well tolerated by patients and provide for rapid onset of a sufficient in vivo concentration of deuterated arachidonic acid to provide for a reduced and stabilized rate of disease progression.

As used herein, the term “maintenance dose” of deuterated arachidonic acid or a prodrug thereof refers to a dose of deuterated arachidonic acid or a prodrug thereof that is less than the primer dose and is sufficient to maintain a therapeutic concentration of deuterated arachidonic acid in the cell membrane of red blood cells and, hence, in the cell membrane of motor neurons, so as to retain a reduced rate of disease progression. In some embodiments, the deuterated arachidonic acid or prodrug thereof is the same compound as used in the loading dose and the maintenance dose.

As used herein, the term “maintenance dose” of deuterated 11,11-D2-linoleic acid refers to a dose of deuterated 11,11-D2-linoleic acid or an ester thereof that is less than the primer dose and is sufficient to maintain a therapeutic concentration of deuterated arachidonic acid in the cell membrane of red blood cells and, hence, in the cell membrane of motor neurons. In some embodiments, the maintenance dose of 11,11-D2-linoleic acid or ester thereof for an adult is about 5 grams per day (e.g., about 4.75 grams when accounting for the ethyl ester which will be removed and a small percent of impurities).

As used herein, the term “periodic dosing” refers to a dosing schedule that substantially comports to the dosing described herein. Stated differently, periodic dosing includes a patient who is compliant at least 75 percent of the time over a 30-day period and preferably at least 80% compliant with the dosing regimen described herein. In embodiments, the dosing schedule contains a designed pause in dosing. For example, a dosing schedule that provides dosing 6 days a week is one form of periodic dosing. Another example is allowing the patient to pause administration for from about 3 or 7 or more days (e.g., due to personal reasons) provided that the patient is otherwise at least 75 percent compliant. Also, for patients who transition from the loading dose to the maintenance dose, compliance is ascertained by both the loading dose and the maintenance dose.

The term “cohort” refers to a group of at least 2 patients whose results are to be averaged.

As used herein, the term “pharmaceutically acceptable salts” of compounds disclosed herein are within the scope of the methods described herein and include acid or base addition salts which retain the desired pharmacological activity and is not biologically undesirable (e.g., the salt is not unduly toxic, allergenic, or irritating, and is bioavailable). When the compound has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound has an acidic group, such as for example, a carboxylic acid group, it can form salts with metals, such as alkali and earth alkali metals (e.g., Na⁺, K⁺, Ca²⁺, Mg²⁺, Zn²⁺), ammonia or organic amines (e.g., dicyclohexylamine, trimethylamine, trimethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

The phrase “excessive amounts of PUFA such as linoleic acid”, “excessive amounts of linoleic acid”, or “excessive linoleic acid intake,” refer to the total intake of linoleic acid in amounts that would reduce the amount of arachidonic acid, including deuterated arachidonic acid, incorporated into the tissue and bioactive pools of the patient.

Neurodegenerative diseases generally are age-related such that the likelihood of being afflicted with such a disease generally increases as you age. The number of such age-related neurodegenerative diseases is expansive and includes, by way of example only, Amyotrophic Lateral Sclerosis (ALS), Alzheimer's Disease (AD), Multiple Sclerosis (MS), Huntington's Disease, Friedreich's Ataxia, tauopathy, optic nerve degradation to mention just a few. An exception to this generality is Infantile Neuroaxonal Dystrophy (INAD) which is afflicts infants as early as 18 months of age and is due to an inborn error of metabolism in PLA2G6 the enzyme that detoxified 15-HpETE-PE.

Each of these diseases has its own etiology evidencing a unique underlying cause of the disease but a common pathology—neuronal death leading to loss of vital functionality and death. Cellular dysfunctionality occurs when one or more cellular perturbations disturb the ability of the cell to retain stasis. When such perturbations continue unabated, the cellular damage caused by the perturbations becomes non-recoverable leading to cell death. One major cause of cellular dysfunctionality, particularly in neurodegenerative diseases, is the generation of oxidized PUFA products in amounts that exceed the ability of regulatory cellular enzymes to neutralize such products. This inability is associated with impairments to these enzymes that can be aged related or genetically related.

As to oxidized PUFA products, cells membranes comprise phospholipids wherein comprises at least one PUFA. Moreover, these phospholipids are stacked together and each is in intimate contact with its adjacent member. The structure of all PUFAs include a cis 1,4-diene system with varying numbers of unsaturation sites and where a bis-allylic methylene group separates the double bonds found at a 1,4-position. Such a structure is represented below showing both the bis-allylic methylene group and the mono-allylic methylene groups.

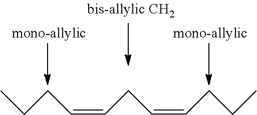

The hydrogen atoms of these bis-allylic methylene groups are particularly susceptible to oxidizing agents and, when oxidized, a cascade of autooxidation occurs where a first oxidized PUFA can initiate oxidation of a neighboring PUFA which, in turn, can initiate oxidation of its neighboring PUFA creating a process cause Lipid Peroxidation (LPO).

The toxicity of oxidized PUFA products generated by LPO is well established. Oxidation occurs in the phospholipid portion of a biological membrane Once oxidized, enzymes such as A2 phospholipase remove the oxidized PUFA followed by neutralization of the oxidized product by a number of endogenous enzymatic antioxidants including superoxide dismutase (SOD), catalase (CAT) and glutathione peroxidase (GPx), and thioredoxin (Trx). Impairment of any of these enzymes can lead to an accumulation of non-neutralized oxidized products which, in turn, causes the toxicity. For example, INAD is caused by genetic defects in the A2 phospholipase enzyme which limits it ability to remove (hydrolyze) oxidized arachidonic acids at the SN2 position of the phospholipid.

As these oxidative perturbations continue, cellular dysfunctionality often occurs prior to cell death. Continuation of these perturbations leads to cell death via a regulated cell death (RCD) pathway. These RCD pathways are well recognized in the art and include, by way of example only, intrinsic apoptosis, extrinsic apoptosis, mitochondrial permeability transition (MPT)-driven necrosis, necroptosis, oxytosis, ferroptosis, and pyroptosis to name a few. In each case, cell death is the result of unrecoverable cellular perturbations that terminate cell survival. See, e.g., Galluzzi, et al., Nature, 25:486-541 (2018).

Oxidized perturbations associated with the accumulation of oxidized PUFA products have been implicated in a wide variety of diseases including mitochondrial diseases, neurodegenerative diseases (including neurodegenerative muscle diseases), retinal diseases, energy processing disorders, cardiac diseases, to name a few.

Enzymatic impairment can be due to genetic defects which limit enzymatic activity below that necessary to neutralize oxidized PUFA products. Alternatively, enzymatic impairment can be age related. In one exemplary embodiment which is but one example of an oxidized products, the amounts of enzymes expressed and/or the activity of these expressed enzymes are reduced as one ages. Still further, enzymatic impairment can also be due to the inability of the cell to produce sufficient enzyme to counter an increasing amount of oxidized arachidonic acid products arising from a diseased condition. Regardless of the cause of such impairment, the impaired enzymes are capable of neutralizing only a fraction of the oxidized products thereby allowing for the accumulation of these products. As the amount of these oxidized products accumulate, the functionality of the cell is first compromised. For example, if the cell is a neuron and the disease is INAD, the loss of cellular functionality is due to impairment of the A2 phospholipase enzyme that correlates to higher and higher intracellular levels of oxidized PUFA products. Neuronal impairment is followed by generating a sufficient amount of 15-HpETE-PE to trigger the death signal. As more and more cells (e.g., neurons) die, there is a corresponding loss of functionality. For example, in the case of INAD and ALS, the death of sufficient number of motor neurons will eventually lead to loss of functionality related to movement and swallowing.

Loss of cellular functionality is followed by cellular death due to a RCD pathway when the cellular perturbation remains unabated. In view of the above, methods that addressed these diseases by addressing the underlying impairment of enzymatic conditions are urgently needed. Preferably, such methods would substantially stop disease progression and then reverse at least a portion of the lost cellular functionality.

Attempts to understand why such divergent etiologies for these diseases would include a common pathology has also led to the understanding that excessive concentrations of 15-HpETE-PE constitute a death signal that triggers the destruction of neurons. See, e.g., Sun, et al. Nature Chemical Biology (2021).

As to this common pathology, there are two plausible theories. Starting with the recent discovery that the brain's natural regulators for 15-hydroperoxy-(Hp)-arachidonoyl-phophatidylethanolamine are Ca+2-independent phospholipases A₂ß (e.g., iPLA₂ß, PLA2G6 or PNPLA9 gene) which hydrolyze (neutralize) peroxidized phospholipids, one can theorizes that as one ages, the ability of Ca+2-independent phospholipases A₂ß to neutralize 15-hydroperoxy-(Hp)-arachidonoyl-phophatidylethanolamine is compromised. In this theory, as one ages, the amount of phospholipids (including lysophospholipids) such as 15-hydroperoxy-(Hp)-arachidonoyl-phophatidylethanolamine, the neurotoxin 15-HpETE-PE, increases as one ages. As the level of these damaged phospholipids increases, it eventually reaches a concentration exceeding the ability of regulatory enzymes like PLA2G6 to effect neutralization. Failure to clear 15-HpETE-PE by PLA2G6 leads to its accumulation in the neuron until it initiates neuronal death. This entire process is referred to as the “death signal” for diseased neurons and is one of the proximate causes of neuronal cell death in a wide range of pediatric and adult neurodegenerative diseases.

An alternative theory presupposes that the level of peroxidized phospholipids generated remains the static but either the expression level of phospholipase iPLA₂ß or the activity of that enzyme is compromised either by genetic or epigentic changes leading to neuronal death. This latter theory would appear consistent with INAD where inborn or genetic defects relating to the expression of PLA2G6, a member of phospholipase iPLA₂ß, are considered to be an underlying etiological event. With this theory it is possible that the carriers of PLA2G6 INAD defects or other acquired or epigenetic defects in PLA2G6 structure or function in neurodegenerative diseases other than INAD leads to reduced clearance of the death signal.

Regardless of the validity of either theory, the common causative factor for each is the generation of peroxidized phospholipids in an amount that cannot be properly regulated by phospholipases iPLA₂ß. In this regard, the generation of reactive oxygen species (ROS) in the brain is known to cause the formation of peroxidized phospholipids including 15-HpETE-PE in the membranes of neurons in vital structures and failure to clear this toxin leads to neuronal death, progression of the disease, loss of vital functions and ultimately death.

While the art has recognized that the in vivo incorporation of deuterated arachidonic acids such as 13,13-D2-arachidonic acid (generated by in vivo conversion of 11,11-D2-linoleic acid) into the neurons stabilizes these neurons against oxidation arising from ROS, such disclosures did not provide any suggestion of regulating the amount of peroxidized phospholipids, in particular 15-HpETE-PE, to a level that inhibits the initiation of the death signal and subsequent destruction of neurons or the levels of this deuterated arachidonic acid necessary to retain vital functionality in the patient.

Thus, regardless of whether the concentration of 15-HpETE-PE that triggers the death signal arises from either a genetic or inborn defect in the ability to regulate the level of peroxidized phospholipids, an acquired defect in the clearance enzyme or because the level of ROS increases as a patient ages or develops a neurodegenerative disease beyond the patient's ability to neutralize the oxidized species generated, there is a need for methods that limit the concentration of 15-HpETE-PE in neurons to avoid loss of vital neurological function and death.

Based on the above, any therapy directed at neurodegenerative diseases mediated at least in part by 15-HpETE-PE should be directed not just to reducing peroxidized phospholipids but must reduce these concentrations to the point that the accumulated amount of 15-HpETE-PE is unable to trigger the neuronal death signal thereby blocking progression of the underlying disease.

### Pathology

The underlying pathology of each of the neurodegenerative diseases is independent of the underlying etiology of the disease. That is to say that whatever divergent conditions trigger some of these neurodegenerative diseases (the etiology), once triggered the pathology of these diseases may involve lipid peroxidation of arachidonic acid in neurons. It should be noted that while deuterated arachidonic acid inhibits lipid peroxidation, there are a number of neurodegenerative diseases that are not treatable by the administration of deuterated arachidonic acid or a prodrug thereof such as Friedrich's Ataxia.

The pathology of these diseases may involve generation of oxidized phospholipids which, in the absence of regulatory processes to neutralize this compound, can through a series of metabolic steps generate neurotoxin 15-HpETE-PE. Upon accumulation of this toxin in the intracellular space of a neuron, it reaches a concentration that signals the cell to die.

The pathology of these diseases may involve the accumulation of oxidized PUFA products in affected neurons. As the amount of oxidized products increases, the affected neurons lose functionality (become dysfunctional) further extending the pathology of the disease. Ultimately, the damage to the cell arising from increasing concentrations of oxidized PUFA products reaches a point where the cell is non-recoverable and the cell initiates a cell death pathway (CDP). There are a number of recognized CDP including intrinsic apoptosis, extrinsic apoptosis, mitochondrial permeability transition (MPT)-driven necrosis, necroptosis, oxytosis, ferroptosis, and pyroptosis as well as initiation of the 15-HpETE-PE death signal in the case of 13-oxidized arachidonic acid products. See, Sun et al., Nature Chemical Biology, 2021 which is incorporated by reference in its entirety.

As per the above, the increasing concentration of oxidized PUFA products in the neurons evidence an inability of the regulatory enzymes to neutralize these products. As the unabated accumulation of the oxidized PUFA products increases within at risk neurons, so too does disease progression. Hence, the methods described herein that entail in vivo delivery of deuterated arachidonic acid thereby limiting the amount of oxidized PUFA products generated, have a positive impact on treating the disease.

Neurodegenerative diseases that respond to the administration of deuterated arachidonic acid are suitable for use in the methods described herein. The methods described herein may comprises reducing the peroxidation of arachidonic acid in phospholipids in at-risk neurons thereby limiting the production of 15-HpETE-PE and the death signal generated thereby. It is understood that treatable neurodegenerative diseases comprise those which are mediated, at least in part, by 15-HpETE-PE. These include amyotrophic lateral sclerosis (ALS), tauopathy (including progressive supernuclear palsy—PSP), Huntington's Disease, Corticobasal disorder (CBD), Frontotemporal dementia (FTD), Nonfluent variant primary progressive aphasia (nfvPPA), APO-e4 Alzheimer's Disease, and late onset Tay-Sachs.

As to the specifics, the discovery of several aldehydes that easily reacted with sulfhydryl groups, resulting in the inhibition of vital metabolic processes, led to the association of polyunsaturated fatty acid peroxidation as a component of the pathology of many of neurodegenerative diseases (Schauenstein, E.; Esterbauer, H. Formation and properties of reactive aldehydes. Ciba Found. Symp. (67):225-244; 1978). Whether as a primary cause of disease or a secondary consequence, such lipid peroxidation is attributed to oxidative stress, which leads to neural death and this implicated in the progression of a number of neurodegenerative diseases.

The origin of the oxidative stress responsible for peroxidation varies due to differences in the underlying etiology. Regardless of the differences in etiology, the production of oxidized PUFA products evidence an imbalance between routine production and detoxification (neutralization) of these oxidized products. The lipid membrane as well as the endoplasmic reticulum and mitochondria of motor neurons are highly enriched in arachidonic acid (a 20-carbon chain polyunsaturated fatty acid (“PUFA”) having 4 sites of cis-unsaturation). Separating each of these 4 sites are 3 bis-allylic methylene groups. These groups are particularly susceptible to oxidative damage due to ROS, and to enzymes such as cyclooxygenases, cytochromes and lipoxygenases, as compared to allylic methylene and methylene groups. Oxidized arachidonic acid is no longer arachidonic acid. Apart from being dysfunctional and leading to further membrane damage, oxidation of arachidonic acid reduces the local concentration of arachidonic acid and must be replaced. Thus, it is a double hit: a positive bioactive membrane component is converted to a toxic membrane component.

Moreover, once a bis-allylic methylene group in one arachidonic acid is oxidized by a ROS, a cascade of further oxidation of other arachidonic acid groups in the lipid membrane occurs. This is because a single ROS generates oxidation of a first arachidonic acid component through a free radical mechanism which, in turn, can oxidize a neighboring arachidonic acid through the same free radical mechanism which yet again can oxidize another neighboring arachidonic acid in a process referred to as lipid chain auto-oxidation. The resulting damage includes a significant number of oxidized arachidonic acid components in the cell membrane.

Given that the neurons have a very concentration of arachidonic acid in their lipid membranes, replacement of damage or lost arachidonic acid in these membranes with deuterated arachidonic acid reinforces these structures in the cell and protects against formation of oxidized lipid prodrugs. For example, once a bis-allylic methylene group in one arachidonic acid is oxidized by a ROS, a cascade of further oxidation of other arachidonic acid groups in the lipid membrane occurs. This is because a single ROS generates oxidation of a first arachidonic acid component through a free radical mechanism which, in turn, can oxidize a neighboring arachidonic acid through the same free radical mechanism which yet again can oxidize another neighboring arachidonic acid in a process referred to as lipid chain auto-oxidation. The resulting damage includes a significant number of oxidized arachidonic acid products in the cell membrane and in the membrane of organelles. However, if an oxidized arachidonic acid is adjacent to the deuterated arachidonic acid, then that deuterated arachidonic acid acts as a chain-reaction terminator.

Oxidized arachidonic acid and other oxidized PUFA products negatively affect the fluidity and permeability of cell membranes in the patient's neurons. In addition, they can lead to oxidation of membrane proteins as well as being converted into a large number of highly reactive carbonyl compounds. The latter include reactive species such as acrolein, malonic dialdehyde, glyoxal, methylglyoxal, etc. (Negre-Salvayre A, et al. Brit. J. Pharmacol. 2008; 153:6-20). The most prominent products of arachidonic acid oxidation are alpha, beta-unsaturated aldehydes such as 4-hydroxynon-2-enal (4-HNE; formed from n-6 PUFAs like LA or AA), and corresponding ketoaldehydes (Esterfbauer H, et al. Free Rad. Biol. Med. 1991; 11:81-128). As noted above, these reactive carbonyls cross-link (bio)molecules through Michael addition or Schiff base formation pathways leading which continues the underlying pathology of the disease. Each of these metabolites derived from oxidized PUFAs are encompassed by the term an “oxidized PUFA product”.

### Disease Progression

When a patient is diagnosed with a specific neurodegenerative disease, the clinician evaluates that patient's rate of disease progression by assessing the patient's loss of functionality in the absence of therapy as described herein. That rate is referred to as the “natural history” of the disease and is typically measured by standardized tests that measure the extent of a patient's functionality over a set period of time. As above, the loss of functionality may relate to the accumulation of oxidized PUFA products that arise from the inability of regulatory enzymes to neutralize these products leading to cell dysfunctionality and subsequent death. The greater the accumulation these products, the greater the loss of functionality.

Without being limited to any theory, the progression of a neurodegenerative disease correlates to a loss of some or all of the functionality in the individual neurons due to accumulation of oxidized PUFAs. As concentration of these oxidized products increases to a non-recoverable level, these diseased neurons will initiate the regulatory cell death process.

The treatment described herein provides for a steady increase in the amount of D2-AA in the patient's cells including the neurons. As part of the monitoring process, periodic blood tests measuring the concentration of D2-AA in red bloods cells are necessary to ensure that the patient is progressing to a therapeutic concentration of from about 12% to about 25% as per above on an acceptable time line. At such concentrations, the extent of disease progression is measured by the reduction in the rate loss of functionality over a set period of time as compared to the Natural History of the patient. The greater the reduction in the rate of loss of functionality, the greater the degree of therapy.

As an example, in the case of ALS, there is a standard test referred to as ALSFRS-R which determines the rate of loss of muscle functionality over time and this is used to measure the rate of disease progression. This test has 12 components each of which are measured on a 0 (worse) to 4 (best) scale. The ability of a drug to attenuate the rate of disease progression evidences its efficacy. Even a modest reduction in the rate of functionality loss has been considered significant. However, the continued loss of functionality evidences the inability of the at risk cells (neurons) to prevent accumulation of oxidized PUFA products.

Included among the several categories in 12-part ALSFRS-R functionality test are those directed to vital functionality such as swallowing and respiratory sufficiency (breathing). As a patient declines, particularly near end stage for the disease, the ability to swallow and breathe become more difficult. This is particularly the case with swallowing as difficulty with swallowing can lead to aspiration of food, saliva, etc. which is associated with an increased likelihood of pneumonia resulting therefrom. As such, patients whose swallowing is sufficiently compromised are placed on feeding tubes rather than risk the likelihood of pneumonia and possible death.

Once therapy with a deuterated arachidonic acid or a prodrug thereof (e.g., 11,11-D2-linoleic acid or ester thereof) is initiated, the buildup of deuterated arachidonic acid (e.g., 13,13-D2-arachidonic acid) in vivo, including the at-risk neurons, is an incremental process limited by both physiology of the patient as well as factors such as the turnover rate of arachidonic acid in the patient. Unlike conventional drug therapy where the drug has a very short half-life in vivo, arachidonic acid has a significantly longer half-life. In addition, only a small amount of linoleic acid (e.g., about 10%), a prodrug of arachidonic acid, is bioconverted to arachidonic acid and not all linoleic acid consumed is necessarily absorbed by the body especially if excessive amounts of PUFAs are consumed. Taken together, such dictates that the incremental buildup of deuterated arachidonic acid may require an extended period of time to achieve the desired protective effect.

On the other hand and as shown in the examples, the concentration of 13,13-D2-arachidonic acid in red blood cells required to achieve therapy in the at risk neurons ranges from about 12% to about 25%, and preferably from about 15% to about 20%, based on the total amount of arachidonic acid found in these cells including the deuterated arachidonic acid. Stated differently and using red blood cells as a proxy for determining therapy, when a patient achieves at least about 12% concentration of 13,13-D2-arachidonic acid in these cells, the accumulation of oxidized arachidonic acid in the neurons is stabilized to a level where the impaired regulatory enzymes are capable of neutralizing a large portion to substantially all of these oxidized products. In turn, this reduces the rate of loss of functionality in the treated patient and, in some cases, acts to restore a portion of the functionality previously loss.

Still further, the degree of incorporation of deuterated arachidonic acid into at-risk neurons of the treated patient cannot be directly measured. Hence, indirect methods for such detection are necessary including those set forth in U.S. Provisional Patent Application Ser. No. 63/177,794 which is incorporated herein by reference in its entirety. In that application, the concentration of deuterated arachidonic acid proxy cells such as red blood cells is used as basis to determine the relative uptake of this deuterated PUFA over time. As shown in the examples, concentrations of deuterated arachidonic acid in red blood cells on the order of about 12% to about 25% and, preferably, about 15% to 20%, of the total amount of arachidonic acid, including deuterated arachidonic acid, has been determined to provide therapeutic results against loss of vital functionality. As loss of vital functionality is associated with the death of patients suffering from neurodegenerative diseases, such therapeutic results in extending the life of patients as compared to those who are not so treated.

Notwithstanding the incremental increase in deuterated arachidonic acid in the patient and given the rapid loss of functionality in patients with such neurodegenerative diseases, the clinician must address the patient's need for rapid onset of therapy to preserve as much functionality for the patient including vital functionality.

Given the rapid loss of functionality in patients with neurodegenerative diseases, any dosing regimen employed should address the patient's need for rapid onset of therapy to preserve as much functionality for the patient. Generally, any therapy for treating such neurodegenerative diseases should be effective as soon as practical and preferably within at least 90 days and more preferably within at least 45 days from start of therapy, and more preferably within a month or less, thereby retaining as much of the patient's functionality as possible and furthermore providing for substantial reductions in the rate of disease progression.

One method is to treat the patient with an excessive amount of deuterated arachidonic acid or a prodrug thereof. Another method that is complementary to the first method but relies on administration of 11,11-D2-linoleic acid or an ester thereof as a prodrug of 13,13-D2-arachidonic acid is to limit the extent of consumption of linoleic acid in the diets of the patients. This maximizes the conversion of 11,11-D2-linoleic acid into 13,13-D2-arachidonic acid.

### Compound Preparation

Deuterated arachidonic acids are known in the art and also can be made by conventional chemical synthesis. In addition, a variety of deuterated arachidonic acids, including D2, D4 and D6-arachidonic acids, are described, for example, in Chistyakov, et al., Molecules, 23(12):3331 (2018) as well as in U.S. Pat. Nos. 10,052,299 and 10,577,304, all of which are incorporated herein by reference in their entireties. Esters of these deuterated fatty acids are prepared by conventional techniques well known in the art. Likewise, 11,11-D2-linoleic acid and esters thereof are known in the art. See, e.g., pubchem.ncbi.nlm.nih.gov/compound/124037379. Esters of these deuterated fatty acids are prepared by conventional techniques well known in the art including the ethyl ester.

### Methodology—13,13-D2-Arachidonic Acid or Prodrugs Thereof

The methods described herein may comprise the administration of deuterated arachidonic acid or a prodrug thereof to a patient to treat neurodegenerative diseases mediated by reactive oxygen species. The methods described herein may comprise the administration of deuterated arachidonic acid or prodrugs thereof to a patient to treat neurodegenerative diseases mediated by 15-HpETE-PE.

### Treatment with Deuterated-Arachidonic Acids or Prodrugs Thereof

In some embodiments, 11,11-D2-linoleic acid or an ester thereof is delivered to a patient so as to biogenerate 13,13-D2-arachidonic acid.

In some embodiments, the deuterated arachidonic acid or prodrugs thereof comprise at least one deuterium at the 13-position of arachidonic acid. In some embodiments, the deuterated arachidonic acid or prodrugs thereof comprise D2-arachidonic acid or prodrugs thereof, D4-arachidonic acid or prodrugs thereof, D6-arachidonic acid or prodrugs thereof, or mixtures thereof, each as defined herein. In an embodiment, the deuterated arachidonic acid or prodrugs thereof comprise D2-arachidonic acid or prodrugs thereof. In an embodiment, the deuterated arachidonic acid or prodrugs thereof comprise D4-arachidonic acid or prodrugs thereof. In an embodiment, the deuterated arachidonic acid or prodrugs thereof comprise D6-arachidonic acid or prodrugs thereof. In an embodiment, the deuterated arachidonic acid or prodrugs thereof comprise a mixture of D2-arachidonic acid or prodrugs thereof, D4-arachidonic acid or prodrugs thereof, and/or D6-arachidonic acid or prodrugs thereof. In an embodiment, the deuterated arachidonic acid or prodrugs thereof deliver D2-arachidonic acid to the neurons. In an embodiment, the deuterated arachidonic acid or prodrugs thereof deliver D4-arachidonic acid to the neurons. In an embodiment, the deuterated arachidonic acid or prodrugs thereof deliver D6-arachidonic acid to the neurons. In an embodiment, a mixture of D2-arachidonic acid, D4-arachidonic acid, and/or D6-arachidonic acid is delivered to the neurons.

In some embodiments, a composition of deuterated arachidonic acid or prodrug thereof is employed and comprises on average at least about 80% of the hydrogen atoms at the bis-allylic sites replaced by deuterium atoms. In some embodiments, the deuterated arachidonic acid or prodrug thereof comprises on average at least about 80% of the hydrogen atoms at the bis-allylic sites replaced by deuterium atoms and no more than about 35% on average of the hydrogen atoms at the mono-allylic sites replaced by deuterium atoms.

In some embodiments, such administration comprises the use of a dosing regimen that includes two dosing components. The first dosing component comprises a primer or loading dose of the deuterated arachidonic acid or a prodrug thereof. The second dosing component comprises a maintenance dose of deuterated arachidonic acid or a prodrug thereof, wherein the amount of the deuterated arachidonic acid or a prodrug thereof in said second dosing component is less than that in the first dosing component.

In some embodiments, the amount of deuterated arachidonic acid delivered to neurons of a patient is titrated so as to achieve a concentration of at least about 12% deuterated arachidonic acid in red blood cells as a proxy for the concentration in neurons. In some embodiments, the amount of deuterated arachidonic acid delivered to neurons of a patient is titrated so as to achieve a concentration of from about 12 to about 25%, and preferably, from about 15 to 20%, in red blood cells within at least 6 months from the start of therapy and preferably within 5 months or 4 months or 3 months from the start of therapy. This minimizes buildup of 15-HpETE-PE in the at-risk neurons thereby protecting against loss of functionality and especially against loss of vital functionality. Evidence that vital functionality is protected and extended is provided in the Examples wherein the results of a clinical study demonstrate that patients on therapy lived significantly longer than those not on therapy.

In an embodiment, the loading dose comprises at least about 0.05 grams of deuterated arachidonic acid or a prodrug thereof per day. In an embodiment, the loading dose for the deuterated arachidonic acid or prodrug thereof ranges from about 0.05 grams to about 2 grams per day, administered on a periodic basis as described herein. In general, the D4-arachidonic acid or prodrugs thereof will require less of a loading dose than the D2-arachidonic acid or prodrugs thereof and the D6-arachidonic acid or prodrug thereof require less of a loading dose than the D6-arachidonic acid or prodrugs thereof. Without being limited to any theory, the ability to reduce the amount of deuterated arachidonic acid or prodrugs thereof with higher levels of deuteration is due to the greater extent of protection against lipid peroxidation in vivo. accorded by the increased levels of deuteration. Still further, the dosing of about 0.0.5 grams to about 2 grams per day is measured by the total amount of deuterated arachidonic acid discounting for impurities and the ester portion of the arachidonic acid ester if an ester prodrug is employed. When so employed, the ester group is readily deacylated in the gastrointestinal track. In embodiments, the loading dose is from about 0.05 grams to about 1.5 grams per day. In embodiments, the loading dose is from about 0.10 grams to about 1.5 grams per day. In embodiments, the loading dose is from about 0.10 grams to about 1.25 grams per day. In embodiments, the loading dose is from about 0.10 grams to about 1 gram per day. In embodiments, the loading dose is from about 0.10 grams to about 0.5 grams per day. The loading dose may be any value or subrange within the recited ranges, including endpoints.

As to the primer dose, the amount of deuterated arachidonic acid or a prodrug thereof employed is designed to provide rapid onset of therapy. Such therapy is measured by a reduction in the disease progression of neurodegenerative diseases as described below. In an embodiment, the primer dose takes into account the various complicating factors, such as the amount of PUFAs consumed by the patient in a given day as well as the general turnover rate of lipids (half-life) in the patient's neurons.

Regarding this last point, the lipid components of neurons are not static but, rather, are exchanged over time and have a finite half-life in the body. In general, only a fraction of the lipids components in the lipids are replaced each day. In the case of neurons, these cells are rich in arachidonic acid. The turnover of arachidonic acid in these membranes occurs from a stable pool of lipids comprising arachidonic acid in the spinal fluid. In turn, this stable pool is replaced and replenished over time by arachidonic acid included in the newly consumed lipids by the patient as part of the patient's diet as well as by biosynthesis of arachidonic acid from linoleic acid. In embodiments, the maintenance dose of deuterated arachidonic acid or prodrug thereof is titrated such that the amount of deuterated arachidonic acid administered matches the rate of secretion from the body.

The choice of a dosing of deuterated arachidonic acid or a prodrug thereof as described herein allows for the rapid accumulation of a sufficient amount of deuterated arachidonic acid in the body to achieve early onset to therapeutic concentrations in vivo. When so achieved, the data in the Examples establish that there is a significant reduction in the rate of disease progression.

In embodiments, the loading dose of the dosing regimen described herein includes sufficient amounts of deuterated arachidonic acid that are absorbed into the patient. Once maximized, the resulting deuterated arachidonic acid accumulates in the body and reaches a therapeutic concentration in the patient within about 10 to 45 days after the start of therapy. During this process, deuterated arachidonic acid is systemically absorbed into the cells of the body including neurons. In embodiments, the loading dose is administered for about 10 to about 50 days. In embodiments, the loading dose is administered for about 15 to about 50 days. In embodiments, the loading dose is administered for about 20 to about 50 days. In embodiments, the loading dose is administered for about 10 to about 45 days. In embodiments, the loading dose is administered for about 15 to about 45 days. In embodiments, the loading dose is administered for about 20 to about 30 days. The length of time may be any value or subrange within the recited ranges, including endpoints.

In embodiments, the loading dose is administered at least 5 days per week. In embodiments, the loading dose is administered at least 7 days per week. In embodiments, the loading dose is administered at least once per week. In embodiments, the loading dose is administered at least once per month.

In embodiments, the maintenance dose of deuterated arachidonic acid or a prodrug thereof comprises no more than 65% of the loading dose. In embodiments, the maintenance dose of deuterated arachidonic acid or a prodrug thereof comprises no more than 60% of the loading dose. In embodiments, the maintenance dose of deuterated arachidonic acid or a prodrug thereof comprises no more than 55% of the loading dose. In embodiments, the maintenance dose of deuterated arachidonic acid or a prodrug thereof comprises no more than 50% of the loading dose. In embodiments, the maintenance dose of deuterated arachidonic acid or a prodrug thereof comprises no more than 45% of the loading dose. In embodiments, the maintenance dose of deuterated arachidonic acid or a prodrug thereof comprises no more than 40% of the loading dose.

In embodiments, the maintenance dose of deuterated arachidonic acid or a prodrug thereof comprises no more than 35% of the loading dose. In embodiments, the maintenance dose of deuterated arachidonic acid or a prodrug thereof comprises no more than 30% of the loading dose.

In embodiments, the maintenance dose is administered at least 5 days per week. In embodiments, the maintenance dose is administered at least 7 days per week. In embodiments, the maintenance dose is administered at least once per week. In embodiments, the maintenance dose is administered at least once per month.

As is apparent, it is not practical to ascertain the concentration of deuterated arachidonic acid in a patient's neurons. This requires that such concentrations be ascertained indirectly by a reporter cell such as a red blood cell, a skin cell, etc. In the case of 13,13-D2-arachidonic acid, at the time a therapeutic result in ascertained, red blood cells are obtained from the patient, the amount of 13,13-D2-arachidonic acid contained in said red blood cells based on the total amount of arachidonic acid present, including 13,13-D2-arachidonic acid is measured. When so evaluated, a concentration of at least about 3% and preferably at least about 5%, and more preferably, at least about 8% of 13,13-D2-arachidonic acid when tested at one (1) month after the start of therapy was found to represent a threshold amount required for therapeutic results in the neurons. When so administered, there is a significant reduction in the progression rate of the neurodegenerative disease being treated.

The methods described herein are also based, in part, on the discovery that the dosing regimen set forth herein provides for rapid uptake or accumulation of deuterated arachidonic acid in the lipid membrane of neurons which then stabilizes these membranes against LPO. As a result, there is a substantial reduction in the progression of the neurodegenerative disease. This is believed to be due to the replacement of hydrogen atoms with deuterium atoms in the deuterated arachidonic acid, rendering the deuterated arachidonic acid significantly more stable to ROS than the hydrogen atoms. As above, this stability manifests itself in reducing the cascade of lipid auto-oxidation and, hence, limiting the rate of disease progression.

In the specific instance of ALS, the reduction in the progression of this disease can be readily calculated by using the known and established rate functional decline measured by the R—ALS Functional Rating Scale-revised after commencement of drug therapy as compared to the rate of decline prior to drug therapy (natural history of decline). As the rate of decline is not perceptible on a day-to-day basis, the functional decline is typically measured monthly and is evaluated over a period of time, such as every 1 to 24 months, such as every 3 months, every 6 months, or annually. The period of time may be any value or subrange within the recited ranges, including endpoints.

As set forth in the examples below, the rate of functional decline is predicated on measuring an individual's, or a cohort's, average for the natural history of disease progression. Next, the individual or cohort average for the functional decline is determined at a period of time such as at 3, 6 or 12 months after initiation of therapy. The rate of decline based on the average of the natural history of the cohort is set as the denominator. The numerator is set as the delta between the rate of the natural history of disease progression and the rate of functional decline after a set period of treatment per this invention. The resulting fraction is the multiplied by 100 to give a percent change. The following exemplifies this analysis.

Cohort A has an average natural history rate of decline in functionality of 28 annualized for a one (1) year period. Six (6) months after initiation of treatment per this invention, Cohort A an annualized average rate of decline in functionality has dropped to 14. This provides a delta of 14 degrees. So, using 14 as the numerator and 28 as the denominator and then multiplying result by 100, one obtains a reduction in the annualized rate of decline of 50 percent.

In general, the methods of this invention provide for an average percent change in reduction in functionality for a cohort of at least 30% and, more preferably, at least about 35%, or at least about 40%, or at least about 45%, or at least about 50%, or at least about 55%, or at least about 60%. In embodiments, the change in reduction of functionality is measured over a time period, for example 1 month to 24 months, e.g., at 3 months, at 6 months, or annually. The rate of decline can be measured over any time period intermediate between 3 months and 1 year.

As noted above, the dosing regimen also addresses the challenge of providing for a dosing regimen that allows for rapid onset to therapeutic concentrations of deuterated arachidonic acid to quickly reduce the rate of disease progression in the patient so as to minimize the additional loss of functionality. It is to be understood that reducing the rate of disease progression correlates to longer periods of retained functionality in the patient and likely a longer lifespan. Accordingly, the faster one reaches such a reduced rate, the better off it is for the patient.

In some embodiments, the methods described herein address this challenge by employing a dosing regimen which delivers deuterated arachidonic acid in amounts sufficient to provide for a therapeutic amount to the neurons. When so incorporated, the deuterated arachidonic acid reduces the degree of LPO which, in turn, effectively limits progression of ALS provided it is administered in appropriate amounts.

### 11,11-D2-Linoleic Acid (or the Ethyl Ester Thereof) as a Prodrug

In some embodiments, the methods described herein comprise the administration of 11,11-D2-linoleic acid or an ester (D2-LA) thereof to a patient suffering from a neurodegenerative disease. In vivo, a portion of the D2-LA is bio-converted to 13,13-D2-arachidonic acid (D2-AA). The accumulation of D2-AA in the body is monitored so ensure that a therapeutic concentration of D2-AA is achieved. Such monitoring includes blood tests to ensure that the patient is accumulating D2-AA consistent with achieving a therapeutic target of a concentration from about 12% to 25% wherein the administration results in a concentration to treat neurodegenerative diseases mediated by reactive oxygen species. If the blood tests evidence insufficient levels of D2-AA are found in the red blood cells based on both the dosing levels and the period of time from start of treatment, the clinician can determine if the dosing should be increased, or if a change from the loading dose to the maintenance dose should be delayed.

In the specific instance of ALS, the reduction in the progression of this disease can be readily calculated by using the known and established rate functional decline measured by the R—ALS Functional Rating Scale-revised after commencement of drug therapy as compared to the rate of decline prior to drug therapy (natural history of decline). As the rate of decline is not perceptible on a day-to-day basis, the functional decline is typically measured monthly and is evaluated over a period of time, such as every 1 to 24 months, such as every 3 months, every 6 months, or annually. The period of time may be any value or subrange within the recited ranges, including endpoints.

As set forth in the examples below, the rate of functional decline is predicated on measuring an individual's, or a cohort's, average for the natural history of disease progression. Next, the individual or cohort average for the functional decline is determined at a period of time such as at 3, 6 or 12 months after initiation of therapy. The rate of decline based on the average of the natural history of the cohort is set as the denominator. The numerator is set as the delta between the rate of the natural history of disease progression and the rate of functional decline after a set period of treatment per this invention. The resulting fraction is the multiplied by 100 to give a percent change. The following exemplifies this analysis.

Cohort A has an average natural history rate of decline in functionality of 28 annualized for a one (1) year period. Six (6) months after initiation of treatment per this invention, Cohort A an annualized average rate of decline in functionality has dropped to 14. This provides a delta of 14 degrees. So, using 14 as the numerator and 28 as the denominator and then multiplying result by 100, one obtains a reduction in the annualized rate of decline of 50 percent.

In some embodiments, 11,11-D2-linoleic acid or ester thereof is administered to a patient as a prodrug of 13,13-D2-arachidonic acid and is delivered in sufficient amounts to generate a concentration of 13,13-D2-arachidonic acid in red blood cells of at least about 12% based on the total amount of arachidonic acid in the red blood cells, including 13,13-D2-arachidonic acid. In some embodiments, the amount of 13,13-D2-arachidonic acid, as well as D4 and D6-arachidonic acid, in red blood cells of the treated patient preferably ranges from about 12% to about 25% and more preferably from about 15% to about 20%.

In all cases, the percent of deuterated arachidonic acid in red blood cells is based on the total amount of arachidonic acid in the red blood cells including deuterated arachidonic acid. As deuteration at the 13-position of arachidonic acid is necessary to inhibit oxidation at this site thereby leading to 15-HpETE-PE, the percent of deuterated arachidonic acid in the red blood cells is independent of whether D-2, D-4, or D-6 arachidonic as recited herein is employed.

In some embodiments and in order to achieve such concentrations, the administration uses a dosing regimen that includes two dosing components. The first dosing component comprises a primer dose of 11,11-D2-linoleic acid or an ester thereof. The second dosing component comprises a maintenance dose of 11,11-D2-linoleic acid or an ester thereof wherein the amount of 11,11-D2-linoleic acid or an ester thereof in said second dosing component is less than that of the first dosing component.

As to the primer dose, the amount of 11,11-D2-linoleic acid or an ester thereof employed is designed to provide rapid onset in the concentration of 13,13-D2-arachidonic acid in the at-risk neurons. As noted above, the lipid components of neurons are not static but, rather, are exchanged over time and have a finite half-life in the body. In general, only a fraction of the lipids components in the lipids are replaced each day. In the case of neurons, these cells are rich in arachidonic acid. The turnover of arachidonic acid in these membranes occurs from a stable pool of lipids comprising arachidonic acid in the spinal fluid. In turn, this stable pool is replaced and replenished over time by arachidonic acid included in the newly consumed lipids by the patient as part of the patient's diet as well as by biosynthesis of arachidonic acid from linoleic acid.

As to the later, the rate of arachidonic acid synthesized is typically rate limited to the extent that there is a maximum amount of arachidonic acid that can be generated in a given day. In turn, only a fraction of the linoleic acid consumed is converted to arachidonic acid with the majority of the linoleic acid remaining unchanged. This limited rate of synthesis of arachidonic acid from linoleic acid results in a slow accumulation of 13,13-D2-arachidonic acid concentration. However, by using a loading dose and limiting the amount of PUFAs otherwise consumed, this conversion rate can be maximized. In adults the loading dose of D2 LA is typically 9 g/day x 1 months followed by 5 g/day maintenance dose thereafter. In infants with INAD the loading dose and maintenance to achieve 12-20% D2 AA is typically 3 g/day for 1 month followed by 2 g/day thereafter.

Given the above, the loading dose of the dosing regimen described herein must include sufficient amounts of 11,11-D2-linoleic acid that are absorbed into the patient so as to maximize the in vivo conversion of 11,11-D2-linoleic acid 13,13-D2-arachidonic acid. Once maximized, the resulting 13,13-D2-arachidonic acid accumulates in the body until it reaches a therapeutic concentration in the patient. During this process, 13,13-D2-arachidonic acid is systemically absorbed into the cells of the body including neurons wherein the rate of which such absorption occurs is based on the exchange rate or turnover rate of lipids in the cell membrane of these motor neurons.

The loading dose described herein provide for rapid onset of a therapeutic concentration of 13,13-D2-arachidonic acid in vivo so as to minimize unnecessary loss of functionality in the treated patients suffering from a neurodegenerative disease. The loading dose is achieved by administering 11,11-D2-linoleic acid or an ester thereof to the patient with a dosing regimen that comprises a primer dose and a maintenance dose wherein, the primer dose in adults comprises the periodic administration of from about 7 to 12 grams of 11,11-D2-linoleic acid or an ester thereof per day until the desired concentration of 13,13-D2-arachidonic acid is achieved. In children, the primer dose comprises about 3-5 grams of 11,11-D2-linoleic acid or an ester thereof.

After completion of the primer dose, a maintenance dose is employed. In some embodiments, the maintenance dose comprises the periodic administration of no more than about 65% of the loading dose of 11,11-D2-linoleic acid or an ester thereof per day to maintain said therapeutic concentration of 13,13-D2-arachidonic acid in vivo.

In some embodiments, said neurodegenerative disease is amyotrophic lateral sclerosis, Huntington's Disease, progressive supernuclear palsy (PSP), APO-e4 Alzheimer's Disease, corticobasal disorder (CBD), frontotemporal dementia (FTD), nonfluent variant primary progressive aphasia (nfvPPA), INAD, other tauopathies, or late onset Tay-Sachs.

In some embodiments, said periodic administration of the loading dose comprises administration of about 9 grams of 11,11-D2-linoleic acid or an ester thereof per day for at least 5 days per week and preferably 7 days a week.

In some embodiments, the periodic administration of the maintenance dose of 11,11-D2-linoleic acid or an ester thereof per day comprises no more than 55% of the loading dose which is administered at least once a month. In another embodiment, the maintenance dose comprises no more than 35% of the loading dose which is administered at least once a month.

In some embodiments, the periodic administration of the maintenance dose is calibrated to be an amount of 11,11-D2-linoleic acid or an ester thereof sufficient to replace the amount of 13,13-D2-arachidonic acid removed from the body taking into account the in vivo conversion of a portion of 11,11-D2-linoleic acid to 13,13-D2-arachidonic acid.

In some embodiments, the methods described herein further comprise restricting the patient's consumption of excessive dietary polyunsaturated fatty acids during administration of said primer and said maintenance doses.

In some embodiments, the deuterated linoleic acid ester is 11,11-D2-linoleic acid ethyl ester.

### 13,13-D2-Arachidonic Acid (or the Ethyl Ester Thereof) as a Prodrug

In some embodiments, a deuterated arachidonic acid or prodrug thereof is employed. The advantage of using such compounds is that there is no need to rely upon the limited in vivo conversion of 11,11-D2-linoleic acid to 13,13-D2-arachidonic acid thereby accelerating the delivery of deuterated arachidonic acid to the at-risk neurons.

In this case, the loading dose of deuterated arachidonic acid takes into account the percent conversion of 11,11-D2-linoleic acid to 13,13-D2-arachidonic acid as well as other factors such as the limited amount of linoleic acid including 11,11-D2-linoleic acid absorbed from the body due to possible excessive intake of PUFAs. As a result, the loading dose comprises at least about 0.05 grams of deuterated arachidonic acid or a prodrug thereof per day. In an embodiment, the loading dose for adults for the deuterated arachidonic acid or prodrug thereof ranges from about 0.05 grams to about 2 grams per day, administered on a periodic basis as described herein. For children the loading dose is about 40% of that for adults or from about 0.02 grams to about 0.8 grams per day.

In embodiments, the loading dose is administered at least 5 days per week. In embodiments, the loading dose is administered at least 7 days per week for up to a month. In embodiments, the loading dose is administered at least once per week. In embodiments, the loading dose is administered at least once per month.

In embodiments, the maintenance dose of deuterated arachidonic acid or a prodrug thereof comprises no more than 65% of the loading dose. In embodiments, the maintenance dose of deuterated arachidonic acid or a prodrug thereof comprises no more than 60% of the loading dose. In embodiments, the maintenance dose of deuterated arachidonic acid or a prodrug thereof comprises no more than 50% of the loading dose. In embodiments, the maintenance dose of deuterated arachidonic acid or a prodrug thereof comprises no more than 40% of the loading dose. In embodiments, the maintenance dose of deuterated arachidonic acid or a prodrug thereof comprises no more than 30% of the loading dose. In embodiments, the maintenance dose of deuterated arachidonic acid or a prodrug thereof comprises no more than 20% of the loading dose.

In some embodiments, the periodic administration of the maintenance dose is calibrated to be an amount of deuterated arachidonic acid or a prodrug thereof sufficient to replace the amount of deuterated arachidonic acid removed from the body.

In some embodiments, the methods described herein further comprise restricting the patient's consumption of excessive dietary polyunsaturated fatty acids during administration of said primer and said maintenance doses.

In some embodiments, the deuterated arachidonic acid ester is 13,13-D2-arachidonic acid ethyl ester.

### Combinations

The therapy provided herein can be combined with other treatments used with neurodegenerative diseases provided that such therapy. In some embodiments, deuterated linoleic acid or an ester thereof (including 11,11-D2-linoleic acid ethyl ester) can be used to supplement or replace deuterated arachidonic acid or a prodrug thereof in the loading dose or the maintenance dose provided that replacement is limited to either the loading dose or the replacement dose but not both. This is due to the fact that a portion of 11,11-D2-linoleic acid is bioconverted (e.g., converted within the body) to 13,13-D2-arachidonic acid. The total amount so converted is a fraction of the amount of 11,11-D2-linoleic acid or ester thereof administered. This fractional conversion allows the clinician to titrate the amount of 13,13-D2-arachidonic acid downward by administering 11,11-D2-linoleic acid or ester thereof. This is particularly the case for the maintenance dose where minimal amounts of 13,13-D2-arachidonic acid may be required as the literature recognizes that the amount of biogenerated arachidonic acid is low. See, e.g., Tallima, et al., J. Adv. Res., 11:33-41 (2018). As to 11,11-D2-linoleic acid or ester thereof, the term “ester thereof” refers to the same term used with regard to deuterated arachidonic acid or prodrugs thereof.

In another embodiment, a combination therapy can employ a drug that operates via an orthogonal mechanism of action relative to inhibition of lipid auto-oxidation. Suitable drugs for use in combination include, but not limited to, antioxidants such as edaravone, idebenone, mitoquinone, mitoquinol, vitamin C, or vitamin E provided that none of these anti-oxidants that are directed to inhibiting lipid auto-oxidation, riluzole which preferentially blocks TTX-sensitive sodium channels, conventional pain relief mediations, and the like.

### Pharmaceutical Compositions

The specific dosing of deuterated arachidonic acid or a prodrug thereof is accomplished by any number of the accepted modes of administration. As noted above, the actual amount of the drug used in a daily or periodic dose per the methods of this invention, i.e., the active ingredient, is described in detail above. The drug can be administered at least once a day, preferably once or twice or three times a day.

This invention is not limited to any particular composition or pharmaceutical carrier, as such may vary. In general, compounds of this invention will be administered as pharmaceutical compositions by any of a number of known routes of administration. However, orally delivery is preferred typically using tablets, pills, capsules, and the like. The particular form used for oral delivery is not critical but due to the large amount of drug to be administered, a daily or periodic unit dose is preferably divided into subunits having a number of tablets, pills, capsules, and the like. In one particularly preferred embodiment, each subunit of the daily or periodic unit dose contains about 1 gram of the drug. So, a daily or periodic unit dose of 9 grams of the drug is preferably provided as 9 sub-unit doses containing about 1 gram of the drug. Preferably, the unit dose is taken in one, two or three settings but, if patient compliance is enhanced by taking the daily or periodic unit dose over 2 or 3 settings per day, such is also acceptable.

Pharmaceutical dosage forms of a compound as disclosed herein may be manufactured by any of the methods well-known in the art, such as, by conventional mixing, tableting, encapsulating, and the like. The compositions as disclosed herein can include one or more physiologically acceptable inactive ingredients that facilitate processing of active molecules into preparations for pharmaceutical use.

The compositions can comprise the drug in combination with at least one pharmaceutically acceptable excipient. Acceptable excipients are non-toxic, aid administration, and do not adversely affect the therapeutic benefit of the claimed compounds. Such excipient may be any solid, liquid, or semi-solid that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Other suitable pharmaceutical excipients and their formulations are described in Remington's Pharmaceutical Sciences, edited by E. W. Martin (Mack Publishing Company, 18th ed., 1990).

The compositions as disclosed herein may, if desired, be presented in a pack or dispenser device each containing a daily or periodic unit dosage containing the drug in the required number of subunits. Such a pack or device may, for example, comprise metal or plastic foil, such as a blister pack, a vial, or any other type of containment. The pack or dispenser device may be accompanied by instructions for administration including, for example, instructions to take all of the subunits constituting the daily or periodic dose contained therein.

The amount of the drug in a formulation can vary depending on the number of subunits required for the daily or periodic dose of the drug. Typically, the formulation will contain, on a weight percent (wt %) basis, from about 10 to 99 weight percent of the drug based on the total formulation, with the balance being one or more suitable pharmaceutical excipients. Preferably, the compound is present at a level of about 50 to 99 weight percent.

In preferred embodiment, the drug is encapsulated inside a capsule without the need for any pharmaceutical excipients such as stabilizers, antioxidants, colorants, etc. This minimizes the number of capsules required per day by maximizing the volume of drug in each capsule.

### EXAMPLES

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims. In these examples, the following terms are used herein and have the following meanings. If not defined, the abbreviation has its conventional medical meaning.
D2-AA=13,13-D2-Arachidonic Acid
AA=Arachidonic Acid
ALSFRS-R=Revised ALS Functional Rating Scale
CNS=Central Nervous System
CSF=Cerebral Spinal Fluid
D2-LA=11,11-D2-Linoleic Acid (aka “drug”)
INAD=Infantile Neuroaxonal Dystrophy
LA=Linoleic Acid
LPO=Lipid peroxidation
PK=Pharmacokinetics
RBC=Red Blood Cells
SAE=Serious Adverse Events
NH=Natural History

### Example 1—Determination of D2-AA Concentrations in RBCs and Spinal Fluid/Neurons in a Single Patient

This example determines the relative concentration of D2-AA in the CSF and in RBCs in order to determine a correlation between these two concentrations. Specifically, a patient was continuously provided with a daily dose of 9 grams of D2-LA ethyl ester (which is 8.64 grams of active discounting for impurities and removal of the ethyl ester) over about a six-month period. Periodic samples of blood and CSF were taken and the concentration of both D2-LA and D-2AA in both the RBCs and the CSF were measured. In all cases, the D2-AA was obtained by deacylation of the ethyl ester of linoleic acid in the gastrointestinal tract followed by conversion of D2-LA in vivo to D2-AA.

TABLE 1

| | | | Ratio of |
|---|---|---|---|
| | Concentration of | Concentration of | D2-LA to |
| Time | D2-LA in CSF | D2-AA in CSF | D2-AA in CSF |
| 1 month | 19.8% | 8% | 2.5:1 |

The results found in Table 1 show that the concentration of D2-AA in the cerebral spinal fluid is already 8% based on the amount of arachidonic acid+deuterated arachidonic acid. As the AA required by neurons is obtained from the CSF, it is reasonable to conclude that the neurons had approximately the same levels of D2-AA as that found in the CSF.

Next, Table 2 shows that the concentration of D2-LA and D2-AA in the RBCs at 3 months and 6 months for the same patient.

TABLE 2

| | | | Ratio of |
|---|---|---|---|
| | Concentration of | Concentration of | D2-LA to |
| Time | D2-LA in RBCs | D2-AA in RBCs | D2-AA in RBCs |
| 3 months | 34.7% | 11.8% | 2.9:1 |
| 6 months | 34.5 | 16.7 | 2.1:1 |

Note here that the concentration of D2-AA in RBC's at 3 months is less than that at 6 months evidencing the incremental increase in D2-AA over time. This suggest that more D2-LA is being converted to D2-AA over time. This suggests that a portion of D2-LA previously administered has accumulated in the patient and acts as reservoir for conversion to D2-AA. If so, then reducing the amount of D2-LA when transitioning from a loading dose to a maintenance dose will have minimal impact on the increase in D2-AA in the period after the transition. Moreover, there is an apparent change in the ratio of D2-LA to D2-AA at 2.9:1 at 3 months which changes to 2.1:1 at 6 months. In some embodiments, the ratio of D2-LA to D2-AA in RBCs at 3 and 6 months is represented as 2.5:1+/−0.4 which corresponds favorably to that found in Table 1.

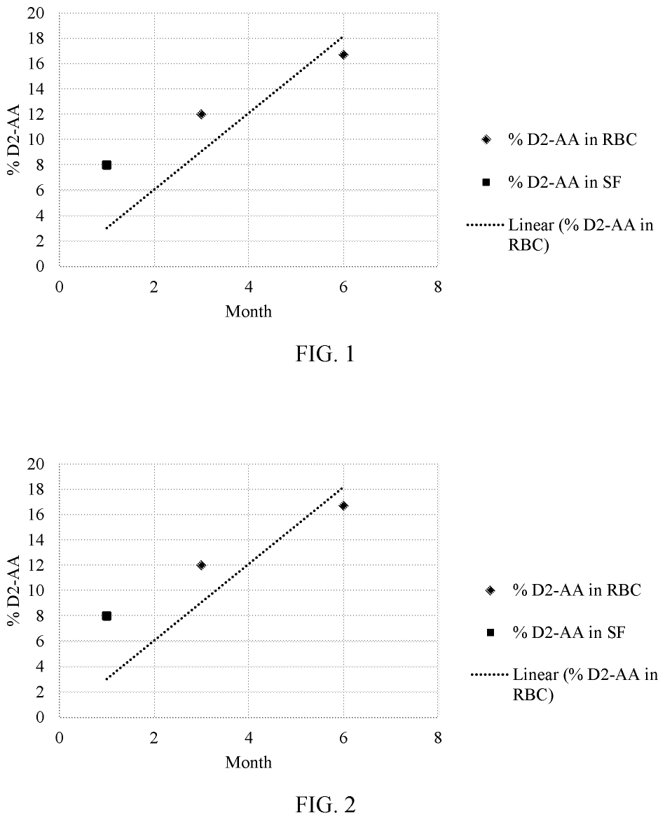

Since the amount of D2-AA is increasing over time in an incremental fashion based on the bioconversion of D2-LA, one can assume a fairly linear rate of increase. This is shown in FIG. 1, where the solid line is set by the concentrations of D2-AA at 3 months and 6 months and then extrapolated back to start of therapy (0 months). The value for the D2-AA in RBC's at 1 month is estimated from this relationship. The amount shown for 1 month in the CSF is also provided (open circle).

Based on the above, one can see that the data to date suggests that the amount D2-AA at 1 month in RBCs would be about 3 percent as compared to 8% for the amount of D2-AA in the SF. Accordingly, this data suggests that the concentration the body shunts more of the AA (including D2-AA) into the CSF (and hence the neurons) as compared to RBCs and likely other reporter cells. Further, taken together with Example 3, the above data demonstrates that a concentration of about 12% (11.8%) in RBCs of this deuterated PUFA correlates to retention of vital functionality.

### Example 2— Determination of AA Concentrations in RBCs and Spinal Fluid/Neurons in a Cohort of 14 Patients

In this example, children suffering from INAD were treated with a daily dose of 3.9 grams of D2-LA ethyl ester followed by 2.9 grams of D2-LA ethyl ester. Given the age and weight of these children, such is assumed to be substantially equivalent to a loading dose of from about 7 and about 12 grams per day for an adult patient for an adult patient and a maintenance dose which is less than the loading dose again for an adult patient.

This example also determines the concentration of D2-AA in RBCs. Specifically, a cohort of 14 children was provided with a daily dose of 3.9 grams of D2-LA ethyl ester for 1 month followed by 2.9 grams of D2-LA ethyl ester for the remaining six-month period. Blood samples were taken at 3 months for all but 1 child and at 6 months for all children. The concentration of D2-AA in RBCs was measured. In all cases, the D2-AA was obtained by deacylation of the ethyl ester of linoleic acid in the gastrointestinal tract followed by bioconversion of D2-LA in vivo to D2-AA.

At 3 months, the average concentration of D2-AA in the RBCs was determined to be 12% (6.8% low and 16.8% high). At 6 months, the average concentration of D2-AA in the RBCs was determined to be 16.7% (12.0% low and 26.1% high). A graph depicting these results is provided as FIG. 2. The line shows a linear relationship of D2-AA accumulation in the body. Included in this graph is the 1-month data for D2-AA in the spinal fluid as found in Example 1.

As can be seen, the graphs in FIGS. 1 and 2 are substantially the same, strongly suggesting that the dosing of D2-LA to the adult patient in Example 1 and to the children in Example 2 maximized the bioconversion of D2-LA to D2-AA. This data further suggests that once maximized, the amounts of D2-AA generated over time are reproducible.

Still further, FIGS. 1 and 2 are representative of a standardized curve evidencing the expected increase in D2-AA in red blood cells from initiation of therapy. The attending clinician can reference such a standardized curve to ascertain whether the patient is progressing to a therapeutic concentration and whether there needs to be any dose adjustments.

### Comparative Example A—The Use of Prodrug of 13,13-Arachidonic Acid

Patients suffering from ALS were treated with D2-LA over a period of time. The patients were given different dosing amounts of D2-LA and for different dosing periods but did not follow the dosing protocol described in U.S. Ser. No. 17/391,909, which is incorporated herein by reference in its entirety. Some patients were provided 2 grams of 11,11-D-2 LA per day as opposed to the loading dose of 9 grams per day.

Functional scores for each of the patients (ALSFRS-R results) at the end of therapy were compared to the natural history scores at the start of therapy. Based on this comparison, the rate of decline changed from an annualized rate of −14.2+/−4.4 per year pre-treatment to −7.6+/−1.4 during treatment or a 46% reduction (p=0.07, paired t-test for within-subject change in slope). When calculated, the amount of D2-AA in the patients' RBCs averaged at about 3% based on the total amount of AA and D2-AA present evidencing that such a concentration provided for therapeutic results.

As D2-LA acts as a pro-drug of D2-AA, the 3% amount of D2-AA in red blood cells shown to be therapeutic would be independent of whether it is delivered by in vivo conversion of D2-LA or by direct administration of D2-AA.

### Example 3—Benefits of the Dosing Protocol Using D2-LA

This example illustrates the reduction in the rate of disease progression in patients with ALS treated by the dosing methods described herein, this example evidences that the amount of oxidized PUFA products has been reduced to a level that allows the otherwise impaired regulatory enzymes are able to neutralize substantially all of these oxidized products now generated. Specifically, a cohort of 3 patients was placed on a dosing regimen consisting of a first dosing component (primer dose) of about 9 grams of D2-LA ethyl ester daily for a period of at least 30 days and then all three patients were transitioned to a second dosing component (maintenance dose) of 5 grams of D2-LA ethyl ester.

The functionality of each of the patients was evaluated periodically using the ALSFRS-R protocol. The patients continued on the dosing regimen for a period of 6 months (patient A) or 1 year (patient B) or for 9 months (patient C). Patient C died at the end of 9 months and his death was attributed to factors other than ALS cardiomyopathy. Before initiation of therapy, the natural history of each patient in the cohort was determined and an average annual rate of functional decline was measured at 21.

The annualized progression of the disease, as measured by an average annual rate of functional decline for all three patients starting at the time that dosing began and terminating at the end of the dosing regimen and then annualized as described above, was measured as 2.1. Using the formula described above, one obtains the following:

(21-2.1)/21×100=90% annualized average reduction in the rate of disease progression.

The specific values for each of the three members of the cohort are as follows in Table 3:

TABLE 3

| | | Functional Rate Decline |
|---|---|---|
| Patient | NH Rate of Decline | During Therapy |
| A | −16 | −3 |
| B | −31 | −2 |
| C | −16 | −1.3 |

These results substantiate a very significant rate of reduction in the disease progression using the dosing regimen as per this invention. These results also substantiate that transitioning patients from a primer dose to a maintenance dose maintains the beneficial stabilization in the rate of decline again suggesting that accumulated D2-LA previously administered to the patient acts as depot as the dosing is changed.

In comparison, patients treated with 9 gm of D2-LA per day for about 1 month followed by 5 gm of D2-LA per day thereafter evidence about a 90% reduction in the rate of disease progression. Compare this to the 46% rate of reduction in the loss of functionality and it is evident that the amount of oxidized product that has not been neutralized is significantly less in this Example as compared to Example 2. This establishes that the dosing regimen described herein provides for a significant benefit to patients in their reduction in the rate of disease progression.

The results of the above examples, demonstrate that when the concentration of 13,13-D2-AA reaches about 12% in red blood cells wherein said concentration is based on the total amount of arachidonic acid present, then the patient's rate of loss of functionality decreases to almost zero.

### Example 4—Survival of Murine Fibroblast Cells in the Presence of Erastin

This example was designed to measure the relative protective activity of 13,13-D2-arachidonic acid as compared to 7,7,10,10,13,13-D6-arachidonic acid in protecting murine fibroblasts from lipid peroxidation mediated cell death. In this example, two different pools of cells were each seeded in 48-well plates and treated with 50 micromolar of erastin. Cells were incubated with either 13,13-D2-arachidonic acid or 7,7,10,10,13,13-D6-arachidonic acid.

Afterwards, cell viability was measured by plate dilution assay to distinguish between cells that are alive and those that are dead on a Petri dish. The results are as follows:

TABLE 4

| Arachidonic acid employed | % Cell Survival |
|---|---|
| 13,13-D2-arachidonic acid | 33.2 |
| 7,7,10,10,13,13-D6-arachidonic acid | 70.2 |

These results evidence that 7,7,10,10,13,13-D6-arachidonic acid provides approximately twice the level of protection against LPO induced cell death as compared to 13,13-D2-arachidonic acid.

### Dosing Based on the Examples

The amount of LA bioconverted to AA is deemed to be in the range of from about 5% to about 30% of the LA consumed. The exact conversion rate depends on factors such as the amount of PUFAs consumed, the amount of AA present in the body coupled with feedback loops, any rate limiting enzymatic steps, and the underlying metabolism of the patient. Therefore, if 2 grams of D2-LA successfully achieves about a 3.0 percent (a therapeutic level) of D-2AA in red blood cells as per Comparative Example A above, and if 15% of the D2-LA (approximately half of 5 to 30 percent) is converted to D2-AA, then one can deduce that:

A. At a 15% conversion rate, the 2 grams of D2-LA would generate about 0.3 grams of D2-AA by bioconversion.

B. At a 30% conversion rate, the 2 grams of D2-LA would generate about 0.6 grams of D2-AA by bioconversion.

Still further, Example 4 illustrates that D6-AA is about 2 times more active than D2-AA. So, when using D6-AA, one can deduce that it will require slightly less than half as much as D2-AA. So, at a low end, the 0.3 grams of D2-AA would translate into about 0.15 grams of D6-AA, or perhaps less. As to the loading dose of D4-AA, it will be intermediate between that for D2-AA and D6-AA.

Still further, to achieve the benefits of Example 3 of a significantly reduced rate of loss of functionality, a dose of 9 grams per day of D2-LA would be required. At a 15% conversion rate, such would translate to 1.45 grams per day of D2-AA. For D6-AA, a reduction by 50% would provide for about 0.75 grams per day.

With the above factors considered, in embodiments, the loading dose of deuterated arachidonic acid or prodrug thereof is expected to range from about 0.01 grams to about 2 grams per day. In a preferred embodiment, dosing is from about 0.05 grams to about 1.5 grams per day. In embodiments, the loading dose is from about 0.10 grams to about 1.5 grams per day. In embodiments, the loading dose is from about 0.10 grams to about 1.25 grams per day. In embodiments, the loading dose is from about 0.10 grams to about 1 gram per day. In embodiments, the loading dose is from about 0.10 grams to about 0.5 grams per day, with preferred dosing ranges of from about 0.1 to about 1.5 grams of deuterated arachidonic acid. Other preferred ranges are provided above.

In embodiments, the maintenance dose of deuterated arachidonic acid or a prodrug thereof comprises no more than about 65% of the loading dose. In some embodiments, the maintenance dose of deuterated arachidonic acid or a prodrug thereof comprises no more than 55% of the loading dose. In some embodiments, the maintenance dose of deuterated arachidonic acid or a prodrug thereof is calibrated to be an amount of deuterated arachidonic acid or a prodrug thereof sufficient to replace the amount of deuterated arachidonic acid eliminated from the body.

### Example 5—Natural History of INAD Patients

This example examines the natural history of 37 patients (infants) suffering from INAD wherein all of the patients were observed for at least one year and some up to 2 years. Novel and conventional developmental neurologic assessment scales appropriate to the disease and which measure vital function in different parts of the brain were applied at base line and at 6 monthly intervals and the health and well-being of the infants were assessed continuously for 1-2 years, including any aspiration pneumonia, hospitalizations and deaths.

In this example, all 37 infants were genetically confirmed to possess a PLA2G6 homozygous or mixed heterozygous enzymopathies. The results of this observation are as follows:

13 serious events of which, 10 were aspiration leading to pneumonias (8 fatal and 2 non-fatal); and

3 additional deaths from other INAD causes.

As to the aspiration pneumonias, these are attributed to reduced capacity to swallow due to loss of vital functionality in the infants. The medical records of these affected individuals in the months before they developed aspiration pneumonia note increased bulbar dysfunction, increased difficulty swallowing, breathing and maintain the integrity of their airway when feeding, drinking or swallowing saliva.

In total, there were 11 deaths in the natural history study with minimum 1 year and up to 2 years of observation of 37 INAD subjects with genetically confirmed PLA2G6 homozygous or mixed heterozygous enzymopathies.

### Example 6—Open Label Treatment Study of INAD Patients

In an open label treatment study, 19 infants were treated for at least 1 year and up to 2.5 years with D2-LA. A portion of this drug is bioconverted into and acts as a prodrug for D2-AA. As is well known, the predominant PUFA in neurons is AA and the in vivo generation of D2-AA over time replaces a portion of the AA in the membrane.

When evaluated, the results of this study evidenced only 4 serious events, 2 non-fatal pneumonia and 2 deaths due to other causes. There were no deaths due to aspiration pneumonia in 19 subjects over 2.5 years treatment with RT001. Table 6 compares the results provided in Example 1 to the results of this Example.

TABLE 5

| | No. of | | | |
|---|---|---|---|---|
| | Aspirations | | | |
| | resulting in | Percent | Fatal | Percent |
| | pneumonias | Pneumonias | Pneumonias | mortality |
| Natural History | 10/37 | 27.0% | 8 | 21.6% |
| (Example 1) | | | | |
| Treatment | 2/19 | 10.5% | 0 | 0% |
| (Example 2) | | | | |

This striking improvement in overall survival and freedom from serious life-threatening events was mirrored by improvement in vital function, such as preserved bulbar function, were there was an observed improvement of X-fold on treatment over the 1st year in these measures versus deterioration in the same measures in the natural history study.

Altogether, this data supports that D2-AA is protective of vital functionality in the treated patients as compared to the non-treated patients thereby evidencing neuronal survival in the treated patients and neuronal death in the natural history. As neuronal death in INAD is associated with a death signal generated by accumulation of 15-HpETE-PE, these results indicate that D2-AA is protective against such a death signal being generated.

### Example 7—Concentration of D2-AA in INAD Treated Patients

In this Example, the average plasma level of D2-AA at 6 months in the 15 infants was measured and was determined to be 17.4%. Such a concentration correlates to a similar concentration in RBCs. As with Example 1, such a concentration correlates to retention of vital functionality.

1. A method for inhibiting cellular dysfunctionality and subsequent cell death due directly or indirectly to cellular accumulation of oxidized PUFA products, comprising incorporating deuterated arachidonic acid into a cell and components thereof in sufficient amounts to reduce the amount of oxidized PUFAs generated to a level that regulatory enzymatic processes are capable of neutralizing more or most of said oxidized products produced thereby inhibiting cellular dysfunctionality and subsequent cell death.

2. The method of claim 1, wherein said enzymatic impairment is due to one or more of: genetic defects leading to enzyme with reduced activity; reduction of the amount of enzyme expressed; a reduction in the activity of the enzyme; inability of the cell to produce sufficient enzyme to counter an increasing amount of oxidized PUFA products arising from a diseased condition; or a combination of two or more of these factors.

3. The method of claim 1, wherein the enzymatic impairment is due to age.

4. The method of claim 1, wherein the cell death is the result of a regulatory cell death pathway.

5. The method of claim 4, wherein the regulatory cell death pathway is selected from the group consisting of intrinsic apoptosis, extrinsic apoptosis, mitochondrial permeability transition (MPT)-driven necrosis, necroptosis, oxytosis, ferroptosis, and pyroptosis.

6. The method of claim 1, wherein the cell death in initiated by the presence of a sufficient amount of 15-HpETE-PE to trigger the cellular death signal.

7. The method of claim 1, wherein the cell is a neuron.

8. The method of claim 1, wherein the deuterated arachidonic acid is more resistant to oxidation at the bis-allylic sites by reactive oxygen species (ROS) as compared to their corresponding wild types.

9. The method of claim 1, wherein incorporating deuterated arachidonic acid results in limiting the increase in concentration of or reducing the concentration of 15-peroxidized arachidonic acid, a precursor of 15-HpETE-PE, in the phospholipids.

10. The method of claim 9, wherein incorporating deuterated arachidonic acid results in limiting the concentration of 15-HpETE-PE present in a neuron to delay or prevent the cell from reaching an intracellular concentration of 15-HpETE-PE below that sufficient to trigger the death signal.

11. A method for restoring at least a portion of cellular functionality lost in dysfunctional cells which method comprises incorporating deuterated arachidonic acid into the cell and components thereof in sufficient amounts to reduce the amount of oxidized PUFA products generated to a level that said impaired enzymatic processes are capable of neutralizing more or most of said oxidized products thereby revitalizing said cell and, upon revitalization, restoring at least a portion of the functionality lost by the cell.

12. A method to treat a neurodegenerative disease in a patient wherein the disease is mediated by neural accumulation of oxidized PUFA products as a result of impaired enzymatic process(es) that limit the amount of the oxidized products that can be neutralized, the method comprises administering a sufficient amount of 11,11-D2-linoleic acid to said patient for a sufficient period of time such that a concentration of 13,13-D2-arachidonic acid in red blood cells ranges from about 12% to about 25% based on the total amount of arachidonic acid including deuterated arachidonic acid, thereby limiting the amount of oxidized PUFA products generated to a level that the impaired enzymatic process(es) are capable of neutralizing substantially all of the oxidized products, thereby treating the disease.

13. The method of claim 12, wherein a concentration of 13,13-D2-arachidonic acid in red blood cells in a blood sample obtained from the patient was assessed.

14. The method of claim 13, wherein the concentration of 13,13-D2-arachadonic acid was obtained at a set period after start of therapy and compared to a control.

15. The method of claim 14, wherein the control is a standardized concentration curve.

16. The method of claim 14, further comprising assessing whether the amount of 11,11-D2-linoleic acid or ester thereof administered to the patient should be changed.

17. The method of claim 16, wherein the amount of 11,11-D2-linoleic acid or ester thereof administered to the patient should be increased if the concentration of 13,13-D2-arachidonic acid in the red blood cells is lower than the control.

18. The method of claim 12, wherein the enzymatic impairment is due to one or more of: genetic defects leading to enzyme with reduced activity; reduction of the amount of enzyme expressed; a reduction in the activity of the enzyme: inability of the cell to produce sufficient enzyme to counter an increasing amount of oxidized PUFA products arising from a diseased condition; or a combination of two or more of these factors.

19. The method of claim 18, wherein the enzymatic impairment is due to age.

20.-27. (canceled)
